(19) United States
(12) Patent Application Publication
(10) Pub. No.: US 2021/0077527 A1
(43) Pub. Date: Mar. 18, 2021

(54) UNIVERSAL DONOR SELECTION METHOD TO IDENTIFY NK-CELL-DONORS

(71) Applicant: The Research Institute at Nationwide Children's Hospital, Columbus, OH (US)

(72) Inventor: Dean Lee, Canal Winchester, OH (US)

(21) Appl. No.: 17/018,681

(22) Filed: Sep. 11, 2020

Related U.S. Application Data
(60) Provisional application No. 62/900,245, filed on Sep. 13, 2019.
(60) Provisional application No. 63/049,325, filed on Jul. 8, 2020.

(51) Int. Cl. A61K 35/17; C12N 5/0783; G01N 33/50

(52) U.S. Cl. CPC A61K 35/17; C12N 2501/2321; G01N 33/505; C12N 5/0646

(57) ABSTRACT

Described herein are compositions comprising universal donor natural killer (NK) cells, populations of such cells, methods of obtaining and preparing such cells, and methods of use of such cells and compositions in medical treatment of cancers and infectious disease.

### CROSS REFERENCES TO RELATED APPLICATIONS

The following application claims priority under 35 U.S.C. § 119(e) to co-pending U.S. Provisional Patent Application Ser. No. 62/900,245 filed Sep. 13, 2019 entitled A UNIVERSAL DONOR SELECTION ALGORITHM TO IDENTIFY NK CELL DONORS WITH IDEAL CHARACTERISTICS FOR ANY RECIPIENT and to co-pending U.S. Provisional Patent Application Ser. No. 63/049,325 filed Jul. 8, 2020 entitled UNIVERSAL DONOR SELECTION METHOD TO IDENTIFY NK-CELL-DONORS.

### TECHNICAL FIELD

The present disclosure relates generally to a donor selection method for natural killer (NK) cells and, more specifically, to provide a method of selecting universal donor cells for therapeutic administration to a recipient in need thereof.

### BACKGROUND

Human natural killer (NK) cells express multiple receptors that interact with Human Leukocyte Antigen (HLA) class I molecules. These NK cell receptors belong to one of two major protein superfamilies, the immunoglobulin superfamily or the C type lectin superfamily. The ability of NK cells to discriminate normal from pathologic self-tissues is largely explained by the inhibitory function of the killer cell immunoglobulin-like receptor (KIR) family which predominantly recognize classical HLA class I molecules on potential targets. This self-Major Histocompatibility Complex (MFIC) recognition confers functional competence on the NK cell to be triggered through their activation receptors, a process termed licensing. As a result, licensed NK cells with self-MHC-specific receptors are more readily activated as compared with unlicensed NK cells without self-MHC-specific receptors. Different KIR family members interact with discrete I ILA class I allotypes and have extensive genetic diversity. Similarly, NK cells simultaneously express multiple different receptors with different specificities. As a result, any attempt to utilize NK cells in an adoptive immunotherapy has to contend with the compatibility between the NK cell donor and recipient. It can be costly and time-consuming testing of multiple donors to identify a specific donor for a specific patient. What is needed is a universal source of NK cells that do not suffer from compatibility issues.

### SUMMARY

In one aspect, the present disclosure relates to a method of selecting universal donor NK cells for therapeutic administration to a subject in need thereof, the method comprising: determining a KIR phenotype of candidate NK cells from an NK cell donor, wherein the KIR phenotype is indicative of the presence of one or more variably inherited inhibitory KIRs 2DL1, 2DL2, 2DL3, and 3DL1; and selecting the candidate NK cells as universal donor NK cells for therapeutic administration when the KIR phenotype indicates the presence of one or more variably inherited inhibitory KIRs 2DL1, 2DL2, 2DL3, and 3DL1.

In another aspect, the present disclosure relates to a method of selecting universal donor NK cells for therapeutic administration to a recipient subject in need thereof, the method comprising: obtaining a HLA genotype of candidate NK cells from an NK cell donor, wherein the HLA genotype is indicative of the presence or absence of at least two HLA C1, C2, and Bw4 alleles, and thereby indicative of the presence of one or more variably inherited inhibitory KIRs 2DL1, 2DL2, 2DL3, and 3DL1; and selecting the candidate NK cells as universal donor NK cells for therapeutic administration when the HLA genotype of the candidate NK cells indicates the presence of at least two of the HLA C1, C2, and Bw4 alleles. The method may further comprise obtaining or having obtained a KIR phenotype of the candidate NK cells, wherein the KIR phenotype is indicative of the presence or absence of activating KIRs selected from the group consisting of 2DS1/2, 2DS3/5, 3DS1, and 2DS4; and further selecting the candidate NK cells as a wherein candidate NK cells comprising at least three activating KIRs 2DS1/2, 2DS3/5, 3DS1, and/or 2DS4 are universal NK cells. The method may further comprise obtaining or having obtained a HLA genotype of candidate NK cells from an NK cell donor, wherein the 1-ILA genotype is indicative of the presence or absence of HLA C1, C2, and Bw4 alleles and thereby indicative of the presence of one or more variably inherited inhibitory KIRs 2DL1, 2DL2, 2DL3, and 3DL1 and further selecting the candidate NK cells as a universal donor NK cell for the therapeutic administration when the HLA genotype indicates the presence of at least two HLA alleles HLA C1, C2, and Bw4.

The present disclosure also relates to a method of selecting universal donor NK cells for therapeutic administration to a recipient subject in need thereof, which method comprises obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4, and selecting the candidate NK cells as a universal donor NK cell for the therapeutic administration when the KIR genotype indicates the presence of at least three activating KIRs 2DS1/2, 2DS3/5, 3DS1, and/or 2DS4. The present disclosure additionally relates to a method of screening a population of candidate NK cells from a donor to identify universal NK donor cells in the population for providing a source of NK cells for therapeutic administration to subjects in need thereof, the method comprising (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2 or 2DL3, and/or 3DL1; wherein candidate NK cells comprising at least two HLA alleles HLA C1, C2, and Bw4 and therefore comprising at least one of the variably inherited inhibitory KIRs 2DL1, 2DL2 or 2DL3, and/or 3DL1 are universal donor NK cells. This method may further comprise obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; wherein candidate NK cells comprising at least three activating KIRs 2DS1/2, 2DS3/5, 3DS1, and/or 2DS4 are universal NK cells. In any of these methods, the selected universal donor NK cells may be histologically optimized for at least 50%-85% of recipient subjects. Any of these methods may also include obtaining or having obtained the CMV seropositivity of the candidate NK cells, wherein the NK candidate NK cells are further selected when the NK cell donor is seropositive for CMV, or the NK cells from the NK cell donor have high NKG2C expression compared to a reference level of NKG2C expression. In one aspect of such a method, the reference level of NKG2C expression is below 5% of NK cells expressing NKG2C. In another aspect of such a method, high NKG2C expression is between 5% to about 22% of NK cells expressing NKG2C.

In another aspect, the present disclosure provides an isolated universal donor NK cell selected by or screened by any of the methods discussed herein, wherein the NK cells are NKG2C+. The isolated universal NK cell may be activated by incubating the universal donor NK cells in vitro in the presence of IL-21. The IL-21 used in the in vitro activation may comprise soluble IL-21, IL-21-expressing feeder cells (FC21), IL-21 plasma membrane particles (PM21s), or IL-21 exosomes (EX21s).

In another aspect, the present disclosure provides a method of treating a cancer or an infectious disease in a subject, the method comprising administering to the subject a donor NK cell selected by any one or more of the methods discussed above, or a donor NK cell screened by any one or more of the methods discussed above; or the isolated universal NK cell discussed by some or all of the methods discussed above.

The present disclosure further relates to a method of treating a cancer or an infectious disease in a subject comprising (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2, 2DL3, and 3DL1; (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; and (c) selecting the candidate NK cells as a universal donor NK cell for the therapeutic administration when (i) the HLA genotype indicates the presence of at least two HLA alleles HLA C1, C2, and Bw4; and (ii) the KIR genotype indicates the presence of at least three activating KIRs 2DS1/2, 2DS3/5, 3DS1, and/or 2DS4. In one aspect, the selected universal donor NK cells may be histologically optimized for at least 50%-85% of recipient subjects. In another aspect, the method may further comprise obtaining or having obtained the CMV seropositivity of the candidate NK cells, wherein the NK candidate NK cells are further selected when the NK cell donor is seropositive for CMV or the NK cells from the NK cell donor have high NKG2C expression compared to a reference level of NKG2C expression. The method may further comprise incubating the selected universal donor NK cells in vitro in the presence of IL-21. The IL-21 used in the in vitro culture may comprise soluble IL-21, IL-21-expressing feeder cells (FC21), IL-21 plasma membrane particles (PM21s), and/or IL-21 exosomes (EX21s). In the method, the cancer may be selected from a cancer of the blood, lung, esophagus, stomach, pancreas, liver, biliary tract, colon, rectum, breast, ovary, cervix uterus, endometrium, kidney, bladder, testes, prostate, larynx, thyroid, brain or skin. In another aspect of the method, the infectious disease may be caused by a pathogen selected from a virus, bacterium or fungus.

The present disclosure moreover relates to a method for preparing a population of universal donor NK cells for therapeutic administration to a subject in need thereof, the method comprising: (a) obtaining an initial population of NK cells from a NK cell donor, wherein the NK cell donor has a genotype indicating the presence of (i) at least two of variably inherited activating KIRs 2DS1/2, 2DS3/5, 3DS1, and/or 2DS4; and (ii) at least one of C1, C2, and Bw4 alleles; and (b) exposing the initial population of NK cells to IL-21 in vitro for a time and under conditions sufficient to expand the initial population of NK cells. In one aspect of the method, the donor genotype may indicate the presence of C1, C2, and Bw4 alleles. In another aspect of the method, step (b) may occur for a time and under conditions to achieve at least one population doubling. In another aspect of the method, the preferred donor may have a CMV seropositive profile indicative of the presence of NKG2C+NK cells. In another aspect of the method, exposing the initial population of NK cells to IL-21 may comprise contacting the NK cells in vitro with at least one of soluble IL-21, IL-21-expressing feeder cells (FC21), IL-21 plasma membrane particles (PM21s) and IL-21 exosomes (EX21s), or any combination thereof. In another aspect of the method, the IL-21 present on feeder cells (FC21), IL-21 plasma membrane particles (PM21s) and IL-21 exosomes (EX21s) may comprise a form of IL-21 selected from (a) an engineered membrane bound form for IL-21, (b) IL-21 chemically conjugated to the surface of FC21, PM21 or EX21, or (c) or IL-21 in solution mixed to be in co-contact with the NK cells. In another aspect of the method, any one of the FC21, PM21 or EX21 may further comprise (a) an NK stimulatory ligand selected from IL-2, IL-12, IL-18, IL-15, IL-7, ULBP, MICA, OX4OL, NKG2D agonists, Delta-1, Notch ligands, NKp46 agonists, NKp44 agonists, NKp30 agonists, other NCR agonists, CD16 agonists; or (b) membrane bound TGF-β. In another aspect of the method, the NK cells may be further exposed to one or more NK stimulatory ligands selected from a group of soluble and/or membrane bound ligands. In yet another aspect of the method, a population of universal donor NK cells may be prepared.

In another aspect, the present disclosure provides a population of NK cells prepared by any one or more of the proceeding methods, wherein the expanded population of NK cells is characterized by increased ability to produce and secrete anti-tumor cytokines of IFNy or TNFa. In another aspect, a population of NK cells prepared by any one or more of the proceeding methods, comprises an expanded population of NK cells which is characterized by increased expression of NKG2D, increased expression of CD16, increased expression of NKp46, and/or increased KIR expression. In one aspect of the method, the IL-21 present on feeder cells (FC21), IL-21 plasma membrane particles (PM21s) and IL-21 exosomes (EX21s) may comprise a form of IL-21 selected from (a) an engineered membrane bound form for IL-21, (b) IL-21 chemically conjugated to the surface of FC21, PM21 or EX21, or (c) IL-21 in solution mixed to be in co-contact with the NK cells. In a method of any preceding aspect, any one of the FC21, PM21 or EX21 may further comprise (a) an NK stimulatory ligand selected from IL-2, IL-12, IL-18, IL-15, IL-7, ULBP, MICA, OX4OL, NKG2D agonists, Delta-1, Notch ligands, NKp46 agonists, NKp44 agonists, NKp30 agonists, other NCR agonists, CD16 agonists; or (b) membrane bound TGF-β. In one aspect, any one of the FC21, PM21 or EX21 further comprise soluble and/or membrane bound stimulatory ligands.

The present disclosure additionally relates to an engineered NK cell or cell line, wherein the NK cells have been transformed to express one or more HLA alleles comprising C1, C2 or Bw4. In an engineered NK cell or cell line of preceding aspect, the NK cells may have been transformed to express CI, C2, and Bw4. In an engineered NK cell or cell line of any preceding aspect, the NK cells may have been further transformed to express of one or more variably inherited activating KIRs comprising 2DS1/2, 2DS3/5, 3DS1, or 2DS4. In an engineered NK cell or cell line of any preceding aspect, the NK cells may have been further transformed to express two or three or more variably inherited activating KIRs comprising 2DS1/2, 2DS3/5, 3DS1, or 2DS4.

Also disclosed are methods and compositions related to universal donor NK cells that can be used for therapeutic administration to a recipient subject in need thereof. In one aspect, disclosed herein are methods of selecting universal donor NK cells for therapeutic administration to a recipient subject in need thereof, the method comprising: (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL I, 2DL2, 2DL3, and 3DL1 and/or (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4 and (c) selecting the candidate NK cells as a universal donor NK cell for the therapeutic administration when (i) the HLA genotype indicates the presence of at least two HLA alleles HLA C1, C2, and Bw4 and/or (ii) the KIR genotype indicates the presence of at least three activating KIRs 2DS1/2, 2DS3/5, 3DS1, and/or 2DS4.

Also disclosed herein are methods of screening a population of candidate NK cells from a donor to identify universal NK donor cells in the population for providing a source of NK cells for therapeutic administration to subjects in need thereof, the method comprising: (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2 or 2DL3, and/or 3DL1 and/or (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; wherein candidate NK cells comprising (i) at least two HLA alleles HLA CI, C2, and Bw4 and therefore comprising at least one of the variably inherited inhibitory KIRs 2DL1, 2DL2 or 2DL3, and/or 3DL1 and/or (ii) at least three activating KIRs 2DS1/2, 2DS3/5, 3DS1, and/or 2DS4 are universal NK cells.

In another aspect, disclosed herein are methods of screening a population of NK cells or methods of selecting universal donor NK cells for therapeutic administration to a recipient subject of any preceding aspect, wherein the selected universal donor NK cells are histologically optimized for at least 50%-85% of recipient subjects.

Also disclosed herein are methods of screening a population of NK cells, or methods of selecting universal donor NK cells for therapeutic administration to a recipient subject of any preceding aspect, further comprising obtaining or having obtained the CMV seropositivity of the candidate NK cells, wherein the NK candidate NK cells are further selected when the NK cell donor is seropositive for CMV, or the NK cells from the NK cell donor have high NKG2C expression compared to a reference level of NKG2C expression.

In another aspect, also disclosed are isolated universal donor NK cells selected or screened by the method of any preceding aspect. The NK cells of any preceding aspect may be NKG2C+. Also disclosed herein are an isolated universal NK cell or cells of any preceding aspect, wherein the NK cell(s) is/are activated by incubating the universal donor NK cell(s) in vitro in the presence of IL-21. In one aspect, the IL-21 used in the in vitro activation comprises soluble IL-21, IL-21-expressing feeder cells (FC21), 1L-21 plasma membrane particles (PM21s), IL-21 exosomes (EX21s), or any combination thereof. In another aspect, disclosed herein are methods of treating, preventing, inhibiting, and/or reducing a cancer, metastasis, or an infectious disease in a subject in need thereof, comprising administering to the subject a donor NK cell selected by or screened by the method of any preceding aspect; or administering to the subject the isolated universal NK cell or cells of any preceding aspect. For example, in one aspect, disclosed herein are methods of treating a cancer or an infectious disease in a subject comprising (a) obtaining or having obtained a HLA genotype of candidate NK cells from an NK cell donor, wherein the HLA genotype is indicative of the presence or absence of HLA C1, C2, and Bw4 alleles and thereby indicative of the presence of one or more variably inherited inhibitory KIRs 2DL1, 2DL2, 2DL3, and 3DL1; (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; and (c) selecting the candidate NK cells as a universal donor NK cell for the therapeutic administration when (i) the HLA genotype indicates the presence of at least two HLA alleles HLA CI, C2, and Bw4; and (ii) the KIR genotype indicates the presence of at least three activating KIRs 2DS1/2, 2DS3/5, 3DS1, and/or 2DS4.

In another aspect, disclosed herein are methods of treating a cancer or an infectious disease of any preceding aspect, wherein the selected universal donor NK cells are histologically optimized for at least 50%-85% of recipient subjects.

Also disclosed herein are methods of treating a cancer or an infectious disease of any preceding aspect, further comprising obtaining or having obtained the CMV seropositivity of the candidate NK cells; and wherein the NK candidate NK cells are further selected when the NK cell donor is seropositive for CMV, or the NK cells from the NK cell donor have high NKG2C expression compared to a reference level of NKG2C expression.

In another aspect, disclosed herein are methods treating a cancer or an infectious disease of any preceding aspect, further comprising incubating the selected universal donor NK cells in vitro in the presence of IL-21. In another aspect, the IL-21 used in the in vitro culture comprises soluble IL-21, IL-21-expressing feeder cells (FC21), IL-21 plasma membrane particles (PM21s), or IL-21 exosomes (EX21s), or any combination thereof. Also disclosed herein are methods for preparing a population of universal donor NK cells for therapeutic administration to a subject in need thereof, the method comprising: (a) obtaining an initial population of NK cells from a NK cell donor, wherein the NK cell donor has a genotype indicating the presence of (i) at least two of variably inherited activating KIRs 2DS1/2, 2DS3/5, 3DS1, and/or 2DS4; and (ii) at least one, two, or all three HLA alleles comprising of C1, C2, and Bw4 alleles; and (b) exposing the initial population of NK cells to IL-21 in vitro for a time and under conditions sufficient to expand the initial population of NK cells.

In another aspect disclosed herein are populations of the NK cells of any preceding aspect, wherein the isolated NK cells are NKG2C+ or CMV seropositive. The method of preparing a population of NK cells, wherein exposing the initial population of NK cells to IL-21 comprises contacting the NK cells in vitro with at least one of soluble IL-21, IL-21-expressing feeder cells (FC21), IL-21 plasma membrane particles (PM21s) and IL-21 exosomes (EX21s). For example, disclosed herein are methods of preparing a population of NK cells, wherein the IL-21 present on feeder cells (FC21), IL-21 plasma membrane particles (PM21s) and IL-21 exosomes (EX21s) comprises a form of IL-21 selected from (a) an engineered membrane bound form for IL-21, (b) IL-21 chemically conjugated to the surface of FC21, PM21 or EX21, or (c) or IL-21 in solution mixed to be in co-contact with the NK cells. In one aspect, any one of the FC21, PM21 or EX21 further comprise (a) an NK stimulatory ligand selected from IL-2, IL-12, IL-18, IL-15, IL-7, ULBP, MICA, OX4OL, NKG2D agonists, Delta-1, Notch ligands, NKp46 agonists, NKp44 agonists, NKp30 agonists, other NCR agonists, CD16 agonists; or (b) membrane bound TGF-β.

In one aspect, disclosed herein is a population of universal donor NK cells prepared by the method of any preceding aspect. In one aspect, the population of NK cells is characterized by increased ability to produce and secrete anti-tumor cytokines of IFNy or TNFa. In one aspect, the expanded population of NK cells is characterized by increased expression of NKG2D, increased expression of CD16, increased expression of NKp46, increased KIR expression.

Also disclosed herein are engineered NK cells or cell lines, wherein the NK cells have been transformed to express one, two or more I ILA alleles comprising CI, C2 or Bw4 (for example an NK cell or cell line that expresses C1, C2, and Bw4) and/or transformed to express of one, two, three, four, five or more variably inherited activating KIRs comprising 2DS1/2, 2DS3/5, 3DS I, or 2DS4.

One aspect of the present invention includes a method of selecting universal donor NK cells for therapeutic administration, the method comprising identifying NK donor cells having HLA genotypes with at least one of C1, C2, and BW3 alleles as HLA donor cells, thereby indicating the presence of one or more variably inherited inhibitory KIRs comprising at least one of 2DL1, 2DL2, 2DL3, and 3DL1s, identify a number of activating KIRs present in the HLA donor cells, responsive to the number of activating KIRs present in the HLA donor cells being over an activating threshold, identify the HLA donor cells as KIR donor cells, identify an NKG2C expression status of the KIR donor cells, and responsive to the KIR donor cells being NKG2C positive, identify the KIR donor cells as therapeutic donor cells.

Another aspect of the present invention includes a method of selecting and engineering universal donor NK cells for therapeutic administration, the method comprising engineering NK donor cells to express HLA genotypes with at least one of C1, C2, and BW3 alleles to generate HLA NK cells, obtaining a KIR genotype of the HLA NK cells, transforming HLA NK cells to express at least three activating KIRs, the three activating KIRs comprising at least one of 2DS1/2, 2DS3/5, 3DS1, and 2DS4, identify a cytomegalovirus (CMV) seropositive status of the NK donor cells, and responsive to the KIR donor cells being CMV seropositive, utilize the KIR donor cells as therapeutic donor cells.

Yet another aspect of the present invention includes a method of selecting, engineering, and preparing universal donor NK cells for therapeutic administration, the method comprising determining if NK donor cells have HLA genotypes with at least one of C1, C2, and BW3 alleles as HLA donor cell, thereby indicating the presence of one or more variably inherited inhibitory KIRs comprising at least one of 2DL1, 2DL2, 2DL3, and 3DL1, responsive to NK donor cells having HLA genotypes with at least one of C1, C2, and BW3 alleles, identifying the NK donor cells as HLA NK cells, identifying a number of activating KIRs present in the HLA donor cells, responsive to the number of activating KIRs present in the HLA donor cells being over an activating threshold, identify the HLA donor cells as KIR donor cells, identify an NKG2C expression status of the KIR donor cells, responsive to the KIR donor cells being NKG2C positive, identify the KIR donor cells as therapeutic donor cells, and stimulating the therapeutic donor cells with irradiated K562 expressing at least one of membrane bound IL-21, 4-1BBL, and IL-2 for a first feeder duration.

Also disclosed herein is a method of preparing a collection of NK cells from a donor comprising (i) determining from one or more donors: (a) an HLA genotype indicative of the presence or absence of HLA C1, C2, and Bw4 alleles thereby indicative of the presence of one or more variably inherited inhibitory KIRs 2DL1, 2DL2, 2DL3, and 3DL1; and (b) a KIR genotype indicative of the presence or absence of activating KIRs selected from the group consisting of 2DS1/2, 2DS3/5, 3DS1, and 2DS4; (ii) selecting from said donors a universal donor NK for the therapeutic administration of NK cells when (a) the HLA genotype indicates the presence of at least two HLA alleles HLA C1, C2, and Bw4; and (b) the KIR genotype indicates the presence of at least three activating KIRs 2DS1/2, 2DS3/5, 3DS1, and/or 2DS4; and (iii) preparing said collection of NK cells from an ex vivo batch of NK-cells of said universal donor. In the method, selection as a universal donor NK cell for the therapeutic administration may further comprise selecting a donor with a CMV-seropositive profile indicative of the presence of NKG2C+NK cells.

In another aspect, disclosed herein is use of any one or more of the following in the manufacture of a medicament for treating cancer or an infectious disease in a subject: a donor NK cell selected by a method of any of preceding aspect, a donor NK cells screened by a method of any preceding aspect, an isolated universal NK cell of any preceding aspect, a population of universal donor NK cells of any preceding aspect, an engineered NK cell or cell line any preceding aspect.

In another aspect, disclosed herein is use of a population of NK cells in the manufacture of a medicament for treating cancer or an infectious disease in a subject wherein the population of NK cells comprises: (i) an HLA genotype comprising at least two HLA alleles selected from HLA C1, C2 and Bw4 indicative of the presence of one or more variably inherited inhibitory KIRs selected from 2DL1, 2DL2, 2DL3, and 3DL1; and (ii) a KIR genotype comprising at least three activating KIRS selected from the group consisting of 2DS1/2, 2DS3/5, 3DS1, and 2DS4. In a use of any preceding aspect, the NK cell or population of NK cells may be histologically optimized for at least 50%-85% of the recipient subjects. In the use of any preceding aspect, the donor of the NK cell or population of NK cells may be seropositive for CMV, or the NK cell or population of NK cells may have a high NKG2C expression compared to a reference level of NKG2C expression. The use of any preceding aspect may comprise culturing the NK cell or the population of NK cells in vitro in the presence of IL-21 prior to the use in treatment. In the use of any preceding aspect, the IL-21 in the in vitro culture may comprise IL-21, IL-21-expressing feeder cells (FC21), IL-21 plasma membrane particles (PM21s), or IL-21 exosomes. In the use of any preceding aspect, the cancer may be selected from a cancer of the blood, lung, esophagus, stomach, pancreas, liver, biliary tract, colon, rectum, breast, ovary, cervix uterus, endometrium, kidney, bladder, testes, prostate, larynx, thyroid, brain or skin. An infectious disease may be one caused by a pathogen selected from a virus, bacterium or fungus. In the use of any preceding aspect, the NK cell or population of NK cells, and/or the donor of the NK cell or population of NK cells may be selected from a set comprising two or more cells, populations and/or donors of which said HLA genotype and said KIR genotype has been determined.

### BRIEF DESCRIPTION OF THE DRAWINGS

The foregoing and other features and advantages of the present disclosure will become apparent to one skilled in the art to which the present disclosure relates upon consideration of the following description of the invention with reference to the accompanying drawings, wherein like reference numerals, unless otherwise described refer to like parts throughout the drawings and in which:

FIG. 1 illustrates an increasing number of activating KIRs is associated with increased lysis of target cells in accordance with one embodiment of the present disclosure;

FIG. 2 illustrates a table with representative data showing the population distribution of KIR genotypes in accordance with one embodiment of the present disclosure;

FIG. 3 illustrates a method of selecting universal donor NK cells for therapeutic administration to a recipient subject in need thereof, in accordance with one embodiment of the present disclosure;

FIG. 4 illustrates a method of engineering NK cells to encode and/or express various alleles, KIRs, and/or receptors, in accordance with one embodiment of the present disclosure;

FIG. 5 illustrates a method of collecting and preparing universal donor NK cells for therapeutic administration to a recipient subject in need thereof, in accordance with one embodiment of the present disclosure;

FIG. 5A illustrates a schematic of KIR typing of donors (top) across the HLA-C1, C2, Bw4 spectrum to assess presence (grey) or absence (black) of KIR genes (bottom);

FIG. 5B illustrates analysis of PBMCs and donor matched NK cells by flow cytometry to determine KIR expression on NK cells. Expression of 2DL2/3, 2DL1 and 3DL1 was evaluated using KIR-specific antibodies REA147/CH-L,143211 and DX9, respectively. The percentage of NK cells expressing each KIR for individual donors is shown;

FIG. 6 illustrates a method of collecting and preparing universal donor NK cells for therapeutic administration to a recipient subject having a first disease type in need thereof, in accordance with one embodiment of the present disclosure;

FIG. 7 illustrates a method of identifying recipients having the first disease type, and providing treatment using universal donor NK cells, in accordance with one embodiment of the present disclosure;

FIG. 8 illustrates utilizing flow cytometry to show that all CMV+ donors have NK cells expressing NKG2C, and the NKG2C expression is increased after expansion; and

FIG. 9 illustrates utilizing mRNA level measurements that NKG2C expression is increased after expansion

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

The apparatus and method components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

### DETAILED DESCRIPTION

Referring now to the figures wherein like numbered features shown therein refer to like elements throughout unless otherwise noted. The present disclosure relates generally to a donor selection method for natural killer (NK) cells and, more specifically, to provide a method of selecting universal donor cells for therapeutic administration to a recipient in need thereof.

In this disclosure, reference will be made to a number of terms which shall be defined to have the following meanings:

“Optional” or “optionally” means that the subsequently described event or circumstance may or may not occur, and that the description includes instances where said event or circumstance occurs and instances where it does not “Primers” are a subset of probes which are capable of supporting some type of enzymatic manipulation and which can hybridize with a target nucleic acid such that the enzymatic manipulation can occur. A primer can be made from any combination of nucleotides or nucleotide derivatives or analogs available in the art which do not interfere with the enzymatic manipulation.

“Probes” are molecules capable of interacting with a target nucleic acid, typically in a sequence specific manner, for example through hybridization. The hybridization of nucleic acids is well understood in the art and discussed herein. Typically a probe can be made from any combination of nucleotides or nucleotide derivatives or analogs available in the art.

The terms “peptide,” “polypeptide” and “protein” are used interchangeably to refer to a polymer of amino acid residues.

The term “sequence identity” as used herein, indicates a quantitative measure of the degree of identity between two sequences of substantially equal length. The percent identity of two sequences, whether nucleic acid or amino acid sequences, is the number of exact matches between two aligned sequences divided by the length of the shorter sequence and multiplied by 100.

An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. 0. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP can be used using the following default parameters: genetic code—standard; filter—none; strand both; cutoff=60; expect 10; Matrix BLOSUM62; Descriptions 50 sequences; sort by=HIGHSCORE; Databases=non-redundant, GenBank+EMBL+DDBJ+PDB+GenBank CDStranslations+Swiss protein+Spupdate+P1R. Details of these programs can be found on the GenBank website. In general, the substitutions are conservative amino acid substitutions: limited to exchanges within members of group 1: glycine, alanine, valine, leucine, and Isoleucine; group 2: serine, cysteine, threonine, and methionine; group 3: proline; group 4: phenylalanine, tyrosine, and tryptophan; group 5: aspartate, glutamate, asparagine, and glutamine.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) can be compared by determining their percent identity.

As various changes could be made in the above-described cells and methods without departing from the scope of the invention, it is intended that all matter contained in the above description and in the examples given below, shall be interpreted as illustrative and not in a limiting sense.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Treat,” “treating,” “treatment,” and grammatical variations thereof as used herein, include the administration of a composition with the intent or purpose of partially or completely preventing, delaying, curing, healing, alleviating, relieving, altering, remedying, ameliorating, improving, stabilizing, mitigating, and/or reducing the intensity or frequency of one or more a diseases or conditions, a symptom of a disease or condition, or an underlying cause of a disease or condition. Treatments according to the invention may be applied preventively, prophylactically, pallatively or remedially. Prophylactic treatments are administered to a subject prior to onset (e.g., before obvious signs of cancer), during early onset (e.g., upon initial signs and symptoms of cancer), or after an established development of cancer. Prophylactic administration can occur for day(s) to years prior to the manifestation of symptoms of a disease or an infection.

I. Selecting Universal Donors

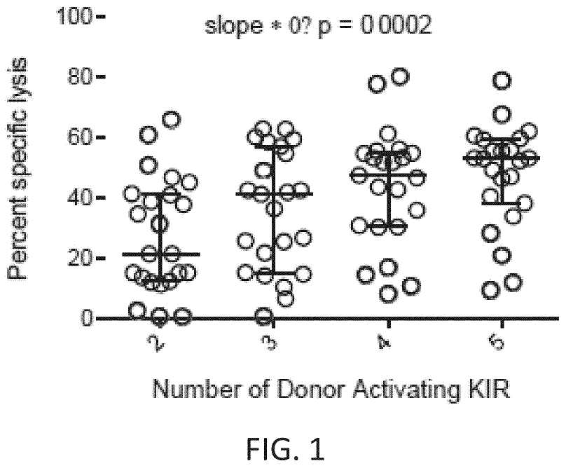

NK cells are licensed (acquire enhanced killing ability) when they express inhibitory killer immunoglobulin receptors (KIR) for self-HLA class I molecules. This enables NK cells to recognize “self” and spare autologous cells from killing. Targets lacking self-HLA class I molecules are thus more likely to elicit recognition by licensed NK cells. The inhibitory KIR genes known to be relevant for NK alloreactivity are: (i) 2DL1 which binds to HLA-C group 2 alleles, (ii) 2DL2 and 2DL3 which bind to HLA-C group 1 alleles, (iii) and 3DL1 which binds to HLA-B Bw4 alleles. According to the missing-ligand model, for each NK cell expressing an inhibitory KIR gene there will be alloreactive only if the corresponding ligand is absent in the recipient, and present in the donor—e.g., any donor possessing a Group C1 allele is alloreactive to any individual lacking a Group C1 allele. Thus, donors who possess HLA in the CI, C2, and Bw4 families are predicted by this model to be alloreactive against any recipient lacking CI, or C2, or Bw4.

Whereas inhibitory KIRs prevent alloreactivity, activating KIRs recognize activating ligands that promote NK cell lysis. Inheritance of activating KIR is widely variable—0 to 7 a KIR are possible in any one individual. Data from patients undergoing stem cell transplantation show that patients receiving allografts from donors with more activating KIRs have a better outcome than patients receiving allograft from donors with fewer activating KIR. Others have shown a protective benefit against leukemia in individuals that inherit more activating KIRs. The laboratory has shown that NK cells with higher numbers of activating KIR induce stronger lysis of target cells (FIG. 1). In addition, the activating KIR 2DS1 and 3DS I are associated with disease-free survival in multivariate analysis.

Lastly, NKG2C is an activating receptor that is expressed late in NK cell development and recognizes HLA-E rather than -B or -C. NKG2C expression is induced in patients with CMV infection and correlates with an adaptive NK cell phenotype and improved leukemia-free survival.

Thus the “universal” donor is one who has an HLA genotype carrying C1, C2, and Bw4 alleles, has a KIR genotype possessing the inhibitory KIR (2DL 1, 2DL2 or 3, and 3DL1) that bind to C1, C2, and Bw4 (leading to maximum licensing) and with a high proportion of activating KIR (>3 of the variably-inherited activating genes including 2DS I and 3DS1), and has been exposed to CMV resulting in high NKG2C expression.

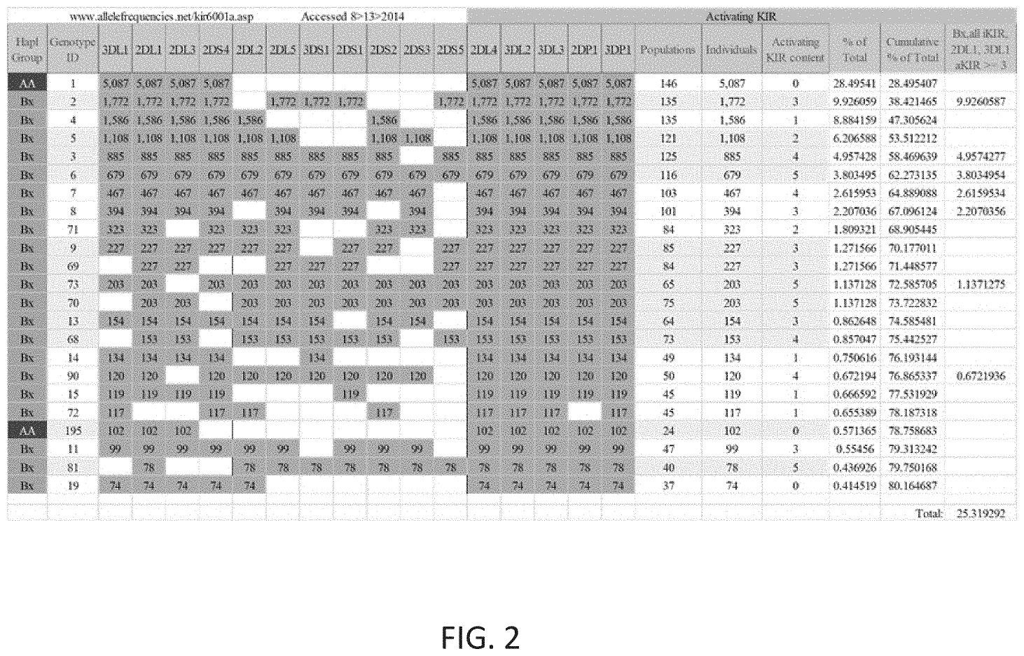

Considering data available for Caucasian donors, C I/C2/Bw4 alleles occur in 32% of the population. Of the 23 KIR genotypes that account for 80% of the population, 25.3% meet all of these criteria (FIG. 2). −90% of adults have been exposed to CMV. Thus, the “ideal” NK cell donor can be identified in approximately 1 out of 16 healthy individuals. It is understood and herein contemplated that by screening for and/or selecting donor NK cells from this 1 out of 16 healthy individuals, a “universal” donor NK cell can be obtained that are histologically optimized for at least 50%-85% of recipient subjects.

Accordingly, in one aspect, the present disclosure relates to a method of selecting universal donor NK cells for therapeutic administration to a subject in need thereof, the method comprising: determining a KIR phenotype of candidate NK cells from an NK cell donor, wherein the KIR phenotype is indicative of the presence of one or more variably inherited inhibitory KIRs 2DL1, 2DL2, 2DL3, and 3DL1; and selecting the candidate NK cells as universal donor NK cells for therapeutic administration when the KIR phenotype indicates the presence of one or more variably inherited inhibitory KIRs 2DL1, 2DL2, 2DL3, and 3DL1. In the method, the KIR phenotype may be determined using image-based methods such as magnetic resonance imaging, which can facilitate high-throughput phenotype imaging. Micro-computed tomographic scanning technology can provide high-precision imaging suitable to support phenotype analysis. Genome-scale RNAi screens can also be applied.

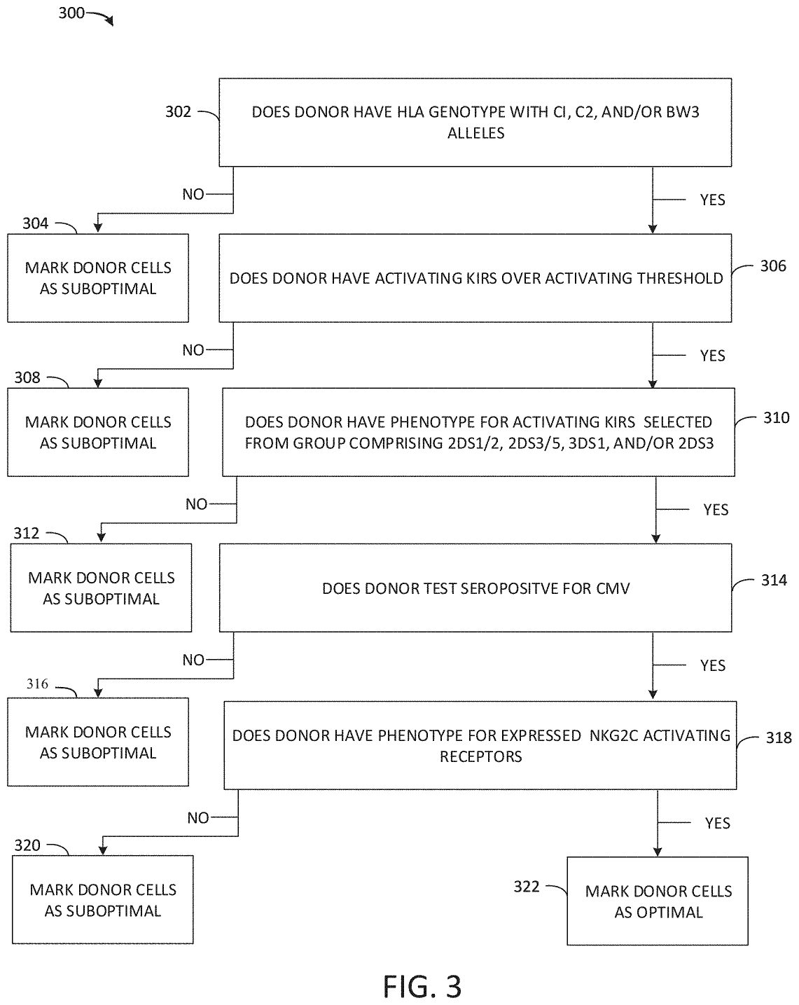

In one aspect, the present disclosure encompasses a method 300 of selecting universal donor NK cells for therapeutic administration to a recipient subject in need thereof, as illustrated in FIG. 3. At 302, it is determined whether the donor cells have HLA C1, C2, and Bw4 alleles. In one aspect, the presence of the HLA C1, C2, and Bw4 alleles is determined by obtaining or having obtained a HLA genotype of candidate NK cells from an NK cell donor, wherein the HLA genotype is indicative of the presence or absence of HLA C1, C2, and Bw4 alleles and thereby indicative of the presence or absence of each of one or more variably inherited inhibitory KIRs 2DL 1, 2DL2, 2DL3, and 3DL1. At 304, responsive to the donor cells lacking at least one of the HLA C1, C2, and Bw4 alleles, the donor cells are marked as sub-optimal. At 306, responsive to the donor cells having at least one of the HLA C1, C2, and Bw4 alleles, it is determined whether donor cells have a number of activating KIRs at or above over an activating threshold, which is a minimum number of activating KIRs present. By way of non-limiting example, in one aspect, the threshold may be at least one activating KIR, wherein the presence of one or more activating KIRs reaches the activating threshold. In alternative aspects, the activating threshold is 2, 3, 4, 5, 6, or 7 activating KIRs, respectively reached when at least one of 2, 3, 4, 5, 6, or 7 activating KIRs are present. In one aspect, the presence of the activating KIRs is determined by obtaining or having obtained a KIR genotype of the candidate NK cells, wherein the KIR genotype is indicative of the presence or absence of activating KIRs. At 308, responsive to the donor cells lacking a number of activating KIRs over the activating threshold, the donor is identified as a non-universal donor.

At 310, responsive to the donor cells having a number of activating KIRs over the activating threshold, determining whether the activating KIRs are selected from a group comprising 2DS1/2, 2DS3/5, 3DS1, and 2DS4. At 312, responsive to the donor cells lacking KIRs selected from a group comprising 2DS1/2, 2DS3/5, 3DS1, and 2DS4, the donor cells are identified as non-universal donor cells.

In one example embodiment, the KIR genotype is indicative of the presence or absence of each of the activating KIRs selected from the group consisting of 2DS1/2, 2DS3/5, 3DS1, and 2DS4. At 314, responsive to the donor being tested for CMV seropositivity, it is determined whether the donor is CMV+. At 316, responsive to the donor testing seronegative for CMV, the donor cells are identified as non-universal donor cells.

At 318, responsive to the donor cells having expressed NKG2C activating receptors, the donor cells are identified as universal donor cells. At 320, responsive to the donor lacking phenotype for expressed NKG2C activating receptors, the donor cells are identified as non-universal donor cells. At 322, responsive to a donor cell satisfying the criteria in at least one, two, three, four or five of steps, 302, 306, 310, 314, and/or 318, a donor cell is identified as a universal donor cell.

In aspect, a donor cell identified as universal is selected as a universal donor NK cell for therapeutic administration to a subject in need thereof. As noted above, NKG2C is an activating receptor that is expressed late in NK cell development and recognizes HLA-E rather than -B or -C. NKG2C expression is induced in patients with CMV infection and correlates with an adaptive NK cell phenotype and improved leukemia-free survival. Thus, identifying candidate donor cells from individuals with elevated NKG2C or that are seropositive for CMV, can further increase the efficacy of the donor NK cells. Thus, also disclosed herein are methods of screening a population of NK cells or methods of selecting universal donor NK cells for therapeutic administration to a recipient subject, where the method further comprises obtaining or having obtained the CMV seropositivity of the candidate NK cells; and wherein the NK candidate NK cells are further selected when the NK cell donor is seropositive for CMV or the NK cells from the NK cell donor have high NKG2C expression compared to a reference level of NKG2C expression. The reference level is for example a predetermined reference value for NKG2C expression obtained from a control donor, or average of NKG2C expression levels obtained from a set of control donors that are seronegative for CMV. It would be understood by one having ordinary skill in the art, that the presence or absence of one of the elements described in 302, 306, 310, 314, and/or 318 does not prevent a donor from ultimately being deemed a universal donor.

In another aspect, the donor is marked optimal when (i) the HLA genotype indicates the presence of at least two I ILA alleles I ILA C1, C2, and Bw4 and/or (ii) the KIR genotype indicates the presence of at least three activating KIRs 2DS1/2, 2DS3/5, 3DS1, and/or 2DS4.

Also disclosed herein are methods of screening a population of candidate NK cells from a donor to identify universal NK donor cells in the population for providing a source of NK cells for therapeutic administration to subjects in need thereof. The method is substantially the same as method 300, except that a population of candidate NK cells are screened. The method of screening a population of candidate NK cells comprising method steps 302-318.

In another aspect, the method of screening a population of candidate NK cells comprises: (a) obtaining or having obtained a HLA genotype of candidate NK cells from an NK cell donor, wherein the HLA genotype is indicative of the presence or absence of HLA CI, C2, and Bw4 alleles and thereby indicative of the presence of one or more variably inherited inhibitory KIRs 2DL1, 2DL2 or 2DL3, and/or 3DL I and/or (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; wherein candidate NK cells comprising (i) at least two HLA alleles HLA C1, C2, and Bw4 and therefore comprising at least one of the variably inherited inhibitory KIRs 2DL1, 2DL2 or 2DL3, and/or 3DL1 and/or (ii) at least three activating KIRs 2DS1/2, 2DS3/5, 3DS1, and/or 2DS4 are universal NK cells.

In one aspect, disclosed herein are methods of screening a population of NK cells or methods of selecting universal donor NK cells for therapeutic administration to a recipient subject of any preceding aspect, wherein the selected universal donor NK cells are histologically optimized for at least 50%-85% of recipient subjects.

It is understood and herein contemplated that the disclosed methods of screening and selecting ultimately produce an isolated universal donor NK cell. Accordingly, disclosed herein are isolated universal donor NK cells wherein the isolated universal donor NK cells comprise at least two I ILA alleles I ILA C1, C2, and Bw4; and/or at least three activating KIRs 2DS1/2, 2DS3/5, 3DS1, and/or 2DS4. In one aspect, the isolated universal donor NK cells are NKG2C+ or derived from a CMV seropositive donor source.

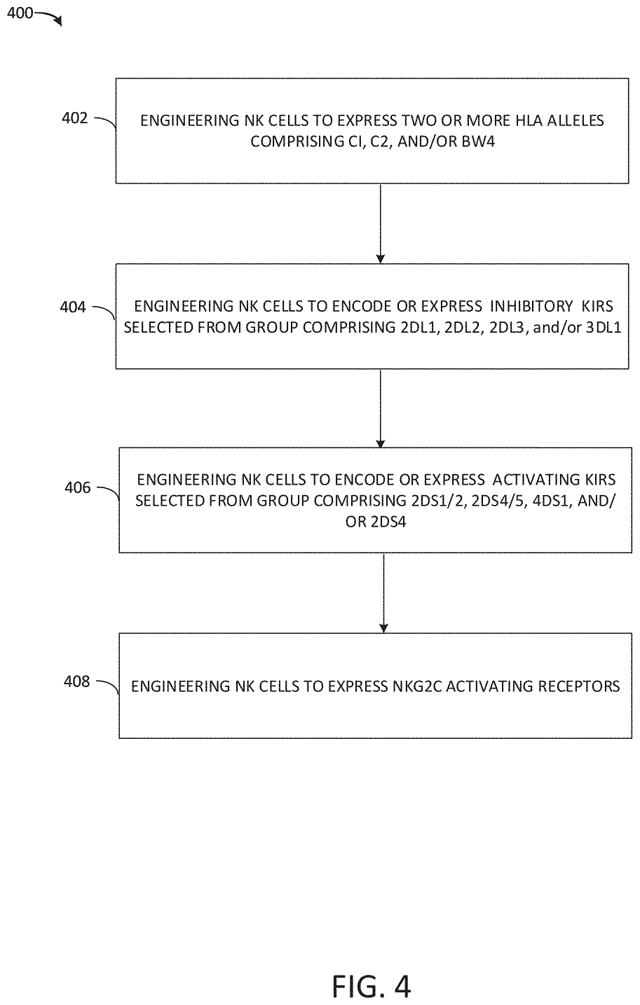

As illustrated in method 400 illustrated in FIG. 4, it is further understood that rather than selecting or screening for the candidate donor NK cells from a donor source to obtain universal donor NK cells with the correct genotype features, NK cells or cell lines can engineered to encode and/or express various alleles, KIRs, and/or receptors. Accordingly, disclosed herein at 402, engineered NK cells or cell lines, wherein the NK cells have been transformed to express one, two or more HLA alleles comprising C1, C2 or Bw4 (for example an NK cell or cell line that expresses CI, C2, and Bw4). At 404, NK cells or cell lines are engineered, wherein the NK cells are transformed to express HLA alleles indicative of the presence of one or more variably inherited inhibitory KIRs 2DL1, 2DL2, 2DL3, and/or 3DL1. At 406, NK cells or cell lines are engineered to encode and/or express activating KIRs 2DS1/2, 2DS3/5, 3DS1, and/or 2DS4. and/or transformed to express of one, two, three, four, five or more variably inherited activating KIRs comprising 2DS1/2, 2DS3/5, 3DS1, or 2DS4. At 408, NK cells or cell lines are engineered to activate NKG2C (e.g., expose cell line to CMV seropositive conditions). Method steps 402-408, may be selectively completed depending upon the underlying gene expression or cell activation present in the NK cell lines being utilized, additionally the steps may be performed on the donor cells marked as suboptimal (e.g., steps 304, 308, 312, 306 of method 300) and/or the donor cells marked as optimal (e.g., step 318 of method 300).

It is understood and herein contemplated that the isolated universal donor NK cells and engineered universal donor NK cells or cell lines can be activated and/or expanded in the presence of one or more NK cell effector agents (e.g., stimulatory peptides, cytokines, and/or adhesion molecules) to overcome many hurdles associated with cytokine toxicity. Examples of NK cell activating agents and stimulatory peptides include, but are not limited to IL-21, 41BBL, IL-2, IL-12, IL-15, IL-18, IL-7, ULBP, MICA, LFA-1, 2B4, BCM/SLAMF2, CCR7, OX4OL, NKG2D agonists, Delta-I, Notch ligands, NKp46 agonists, NKp44 agonists, NKp30 agonists, other NCR agonists, CD16 agonists; and/or TGF-β and/or other homing inducing signaling molecules. Examples of cytokines include, but are not limited to, IL-2, IL-12, 1L-21, and IL-18.

Examples of adhesion molecules include, but are not limited to LFA-1, MICA, BCM/SLAMF2. These NK cell effector agents can be soluble presented in solution or present as membrane bound agent on the surface of plasma membrane (PM) particles, exosome (EX), or feeder cells (FC). The PM particles, EX exosomes, and/or FC cells can be engineered to express membrane forms of the NK cell activating agents and stimulatory peptides. Alternatively, the NK cell activating agents and stimulatory peptides can be chemically conjugated to the surface of the PM particle, EX exosome, of FC feeder cell. For example, a plasma membrane (PM) particle, Feeder cells (FC), or exosomes (EX) prepared from feeder cells expressing membrane bound IL-21 (FC21 cells, PM21 particles, and EX21 exosomes, respectively). Thus, in one aspect, disclosed herein are isolated universal donor NK cells or cell lines wherein the universal donor NK cell or cell line is activated and/or expanded by incubating the universal donor NK cells in vitro in the presence of one or more activating agents, stimulatory peptides, cytokines, and/or adhesion molecules including, but not limited to 41BBL, IL-2, IL-12, IL-15, IL-18, IL-7, ULBP, MICA, LFA-1, 2B4, BCM/SLAMF2, CCR7, OX4OL, NKG2D agonists, Delta-1, Notch ligands, NKp46 agonists, NKp44 agonists, NKp30 agonists, other NCR agonists, CD16 agonists; and/or TGF-β (for example, IL-21). In one aspect, the IL-21 used in the in vitro activation comprises soluble IL-21, IL-21-expressing feeder cells (FC21), IL-21 plasma membrane particles (PM21s), or IL-21 exosomes (EX21s). It is understood and herein contemplated that the membrane bound IL-21 expressing FC21 cells, PM21 particles, and EX21 exosomes may further comprise additional one or more activating agents, stimulatory peptides, cytokines, and/or adhesion molecules including, but not limited to 41BBL, IL-2, IL-12, IL-15, IL-18, IL-7, ULBP, MICA, LFA-1, 2B4, BCM/SLAMF2, CCR7, OX4OL, NKG2D agonists, Delta-1, Notch ligands, NKp46 agonists, NKp44 agonists, NKp30 agonists, other NCR agonists, CD16 agonists; and/or TGF-β (for example, PM21 particle, EX21 exosome, or FC cell expressing 41BBL and membrane bound interleukin-21). NK cells can be additionally exposed to additional ligands both soluble and membrane bound.

As noted above, the additional activation and/or expansion of the universal donor NK cells increases the efficacy of the cell when administered to a recipient. Thus, in one aspect, disclosed herein are methods for preparing a population of universal donor NK cells for therapeutic administration to a subject in need thereof, the method comprising: (a) obtaining an initial population of NK cells from a NK cell donor, wherein the NK cell donor has a genotype indicating the presence of (i) at least two of variably inherited activating KIRs 2DS1/2, 2DS3/5, 3DS1, and/or 2DS4; and (ii) at least one, two, or all three HLA alleles comprising of C1, C2, and Bw4 alleles; and (b) exposing the initial population of NK cells to one or more activating agents, stimulatory peptides, cytokines, and/or adhesion molecules including, but not limited to 11-21, 41BBL, IL-2, IL-12, IL-15, IL-18, IL-7, ULBP, MICA, LFA-1, 2B4, BCM/SLAMF2, CCR7, OX4OL, NKG2D agonists, Delta-1, Notch ligands, NKp46 agonists, NKp44 agonists, NKp30 agonists, other NCR agonists, CD16 agonists; and/or TGF-β (for example, IL-21) in vitro for a time and under conditions sufficient to expand the initial population of NK cells. It is understood and herein contemplated that the exposure to the one or more activating agents can occur for a time and under conditions to achieve at least one population doubling. In one example embodiment, the expansion increases a high NKG2C expression from between 5% to about 22% to between 11% to about 30% of NK cells expressing NKG2C.

In one aspect, the isolated universal donor NK cell or cell line or population of NK cells is characterized by increased ability to produce and secrete anti-tumor cytokines of IFNy or TNFa. In one aspect, the expanded population of NK cells is characterized by increased expression of NKG2D, increased expression of CD16, increased expression of NKp46, increased KIR expression.

II. Donor Selection

In one aspects, donors are screened in step-wise method excluding donors from further testing who do not meet criteria (see FIG. 3). KIR genotyping can be first performed for NK cell donors with reverse sequence-specific oligonucleotide (SSO) methodology (e.g., One Lambda), including discrimination of Functional vs. Deletion variants of KIR2DL4. KIR-B content can be determined using the B Content Calculator maintained by EMBL-EBI. In another example embodiment, activating KIR content is determined by scoring the total number of activating KIR genes. All DS-designated KIR and Functional KIR2DL4 are considered activating. In one aspect, donors are selected who have the common activating KIRs (KIR2DS4 and the functional version of KIR2DL4) and a high number of the 5 variably-inherited activating KIRs. In another aspect, donors are selected on based on the number of B-KIR segments inherited (e.g., 3 or 4 of the centromeric and telomeric B alleles). In one example embodiment, the high number is 3, 4, or 5 of the variably inherited activating KIRs. In another example embodiment, the high number is 4 of the variably inherited activating KIRs. In yet another example embodiment, the high number is having 1 or more of the variably inherited activating KIRs.

In one aspect, NK cell donors arc HLA typed at intermediate or high-resolution level for alleles at HLA-B and -C loci by SSO-PCR (amplification and oligonucleotide sequencing) using commercial kits. In another aspect, KIR-ligand class are predicted using the KIR Ligand Calculator maintained by the European Bioinformatics Institute of the European Molecular Biology Labs (EMBL-EBI). Individuals possessing all three C1, C2, and Bw4 classes are selected.

In one aspect, donors are lastly tested for CMV. CMV+ donors can be tested to confirm the presence of NKG2C+NK cells. Alternatively, donors are screened for the presence of NKG2C+NK cells above the threshold (e.g., −20%) that predicts prior CMV exposure.

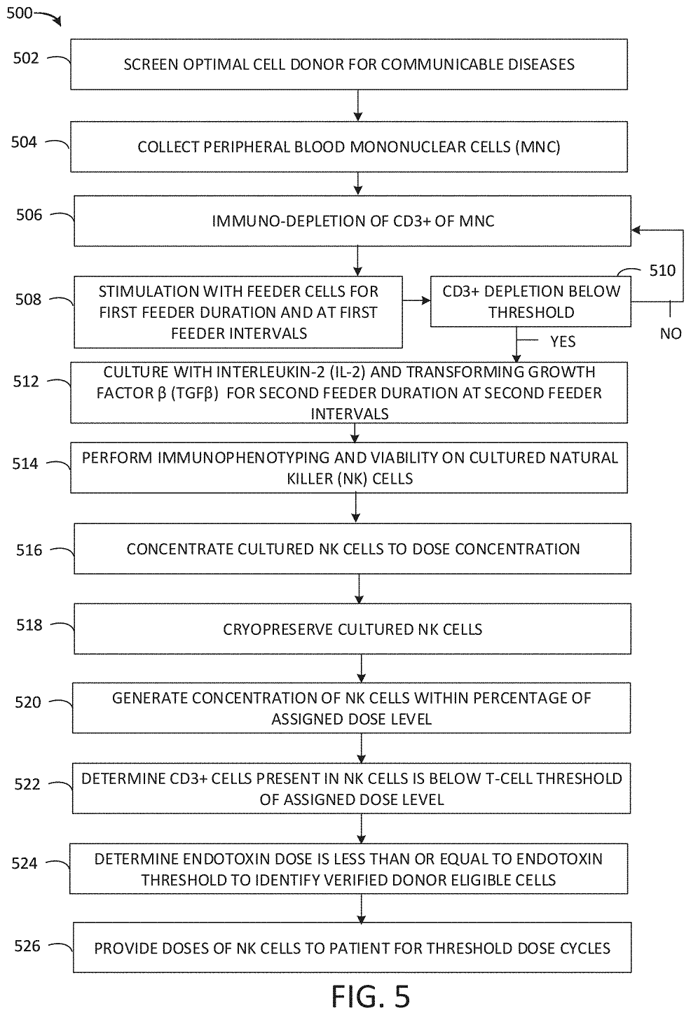

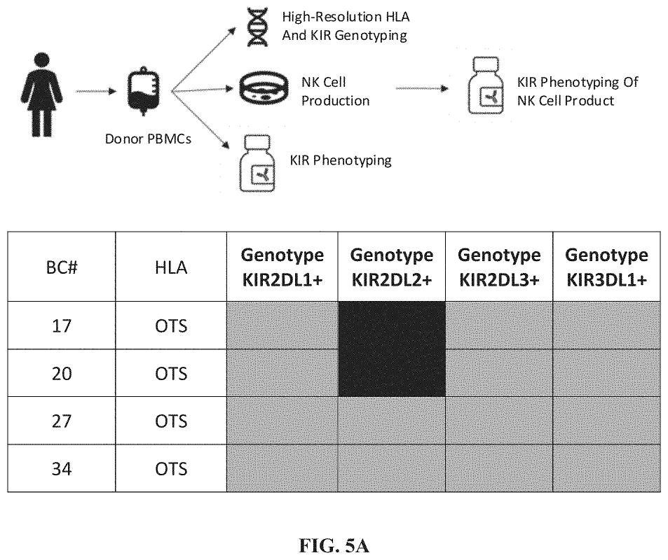

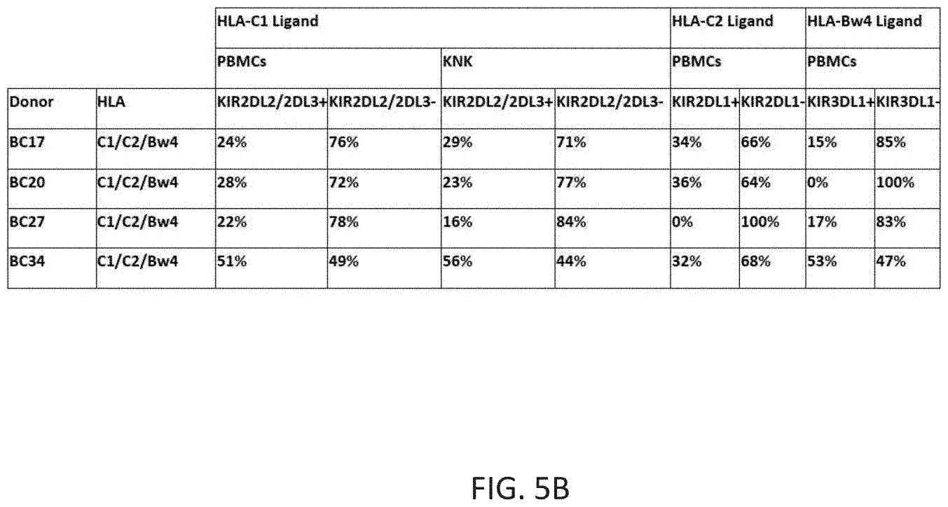

In one aspect, disclosed herein is a method 500 of screening optimal universal donor NK cell donors and preparing the optimal universal donor NK cells for use in various disease treatments as illustrated in FIG. 5. For example, at step 502, optimal cell donors (as defined by method 300 of FIG. 3) are screened for communicable diseases. In this example embodiment, the optimal universal donor NK cell donors (donors) will undergo infectious disease testing and screening as required for HCT/P donors at BTMB institutions compliant with 21 C.F.R. Part 1271, the FDA Guidance document “Eligibility Determination for Donors of Human Cells, Tissues, and Cellular and Tissue-Based Products (HCT/Ps) and any supplemental guidance documents issued. Testing is performed according to BTMB policies and a separate donor protocol utilizing FDA-approved tests for HCT/P donors in CLIA-certified and FDA-registered laboratories under contract with BTMB. Donors will be tested for infectious disease markers (IDMs), using the analytes/test methodology in Table 1, within 7 days before or after collection. The IDMs include Hepatitis B virus Hepatitis C virus, HTLV-I and II, HIV-1, -2, and -O, Syphilis, Trypanosoma cruzi (Chagas Disease), West Nile Virus, CMV.

TABLE 1
Analytes/Test Methodology

| Infectious disease marker | Analyte/Methodology |
|---|---|
| Hepatitis B Virus (HBV) | |
| HBsAg | Hepatitis B surface antigen |
| | screening test |
| Anti HBc Total | Hepatitis B core antibody, |
| | IgG + IgM |
| HBV NAT | Nucleic acid test/PCR |
| Hepatitis C Virus (HCV) | |
| Anti-HCV | Hepatitis C antibody |
| HCV NAT | Nucleic acid test/PCR |
| Human Immunodeficiency virus (HIV) | |
| Anti-HIV 1 and anti-HIV 2 + O | HIV antibodies |
| HIV-1 NAT | Nucleic acid test/PCR |
| Syphilis | Rapid Plasma Reagin test |
| | (Detects Antibodies) |
| West Nile Virus - NAT | Nucleic acid test/PCR |
| Chagas (Trypanasoma Cruzi) | Antibody |
| Cytomegalovirus (CMV) | |
| Anti-CMV Total | CMV antibody, IgG + IgM |

III. Manufacturing and Vialing Estimates

In one aspect, the expanded donor NK cell product is manufactured prior to or in response to patient need. In one aspect, donors undergo standard infectious disease screening and other donor screening (as required by 21 C.F.R. § 1271 subpart C) within 7 days of collection. At step 504 of method 500, responsive to the donor lacking IDMs, Peripheral Blood Mononuclear Cells (MNCs/PBMCs) are collected from the donor. In another example embodiment, Source Peripheral Blood Mononuclear Cells (PBMCs) are collected and NK cells propagated as per standard methods. At 506, the collected MNCs are immune-depleted of CD3+ to form depleted MNCs. In one example embodiment, MNCs/PBMC are depleted of CD3+ T cells using MACS colloidal super-paramagnetic CD3 MicroBeads.

At 508, the depleted MNCs are simulated with feeder cells for a first feeder duration and a first feeder interval to prorogate and activate NK cells. In one example embodiment, the feeder cells are irradiated feeder cells (IFC). In another example embodiment, the depleted MNCs are propirated by recursive weekly stimulation with irradiated CSTX002 feeder cells (cryopreserved or fresh). The CSTX002 is treated with 100 Gy (10,000 rads) gamma-irradiation either (i) prior to cryopreservation or (ii) fresh prior to their addition to MNC or NK cell cultures. Validation of irradiation demonstrates elimination of detectable proliferation at 25 Gy, and co-culture with NK cells provided an additional 99.9% effective elimination of IFC. In one example embodiment, IFCs are added at an approximate 1:2 TNC-to-IFC ratio in media containing RPMI-1640, 10% FBS, 2 mM Glutamax and recombinant human IL-2 (Proleukin, Promethius) at 100 IU/mL. In this example embodiment, the first feeder duration is between 10 to 15 days, and the first feeder interval is 1-5 days. In another example embodiment, the first feeder duration is 14 days, and the first feeder interval is 1-3 days. In one example embodiment, the MNCs or NK cells are re-stimulated with IFCs at an approximate 1:1 TNC-to-IFC ratio and cultured for 7 days (e.g., days 8-14). In this embodiment, the first feeder interval is utilized, wherein in 1-3 day intervals during days 8-14 of expansion, cultures are monitored for cell counts and fresh IL-2 is added at 100 IU/mL and 10 ng/mL of TGF-β. NK cell cultures are split to below 5-10×10⁶ cells per cm² to prevent overgrowth and maximize yield. If needed depending on the culture vessel, fresh media is also provided by at least one half media exchange.

At 510, the CD3+ depletion is determined. Responsive to the CD3+ depletion being above a threshold, step 506 is repeated. In one example, CD3+ depletion is determined a day prior to the end of day 6 of the stimulation of the feeder cells. In this embodiment, samples for cell count, immunophenotyping and viability are obtained from the MNCs and/or NK cell culture (e.g., being stimulated with the feeder cells). In one example embodiment, the threshold of CD3+ depletion is greater than 5% CD3+ cells present. Wherein, in one example embodiment, repeating step 506 includes performing a second cycle of CD3+ depletion on day 7 for the first feeder duration. After the depletion, samples for cell counts, immunophenotyping, and viability will be obtained from the CD3-negative NK cell fraction.

At 512, responsive to the CD3+ depletion being below the threshold, the MNCs and/or NK cells are cultured with interleukin-2 (IL-2) and/or Transforming growth factor β(TGFβ) for a second feeder duration at second feeder intervals. In one example embodiment, the second feeder duration is between about 5-8 days and the second feeder interval is between about 1-5 days. In another example embodiment, the second feeder duration is 7 days and the second feeder interval is between about 1-3 days. In that example embodiment, fresh IL-2 is added at 100 IU/mL and 10 ng/mL of TGF-β is added at the second feeder interval during the first seven days of the first feeder duration.

At 514, immunophenotyping and viability on the cultured natural killer cells are performed. In one example, on day 13 of the first feeder duration, samples for cell count, immunophenotyping and viability are obtained from the NK cell culture. Responsive to less than 0.33% CD3+ cells being present, testing may be repeated immediately or prior to harvest on day 14 of the first feeder duration. Responsive to CD3+ depletion being over a second threshold (e.g., 0.33%), an additional depletion as described at step 506 is performed immediately on day 13 or following harvest on day 14. Samples for cell counts and immunophenotyping and viability are obtained from the CD3-depleted NK cell fraction and the remainder will be returned to culture with IL-2 and TGF-β overnight. In one example embodiment, responsible to no CD3+ depletion being performed, then day 7 immunophenotyping will not be performed.

At 516, the cultured NK cells are concentrated into a dose concentration. In one example, the dose concentration is between 2×10⁶ NC/mL and 2×10⁸ NC/mL. At 518, the cultured NK cells at the dose concentration are cryopreserved. In one example embodiment, the NK cells are cryopreserved in NK Freeze Media. In example embodiment, NK Freeze Media comprises 10% DMSO, 12.5% (w/v) human serum albumin (HSA), USP, and/or In Plasma-Lyte A (USP). Method 500 recites methods of treatment for a particular patient starting at 520, which is continued in detail below.

In one example embodiment, such as when treating a HSV patient having herpes simplex virus (HSV), the HSV patient (person diagnosed with HSV) receives up to 5 consecutive, once daily doses of banked NK cells, dosed at 5.0×10⁷ cells/kg/dose. In this example embodiment, HSV patients with prior transfusion or infusion reactions are pre-medicated with diphenhydramine 1 mg/kg (max 50 mg) IV and acetaminophen 10 mg/kg (max 650 mg) PO. HSV patients undergo repeat eligibility evaluation on subsequent days (D1-D4) to determine if eligible for repeated doses. Doses are given once daily, on 5 consecutive days to HSV patient.

In another example embodiment, such as when treating a COVID patient (e.g., a person with a COVID-19 infection or SARS-COV-2), NK cells are provided as treatment. In one example embodiment, patients with prior transfusion or infusion reactions are pre-medicated with diphenhydramine 1 mg/kg (max 50 mg) IV and acetaminophen 10 mg/kg (max 650 mg). In another example embodiment, patients receive their first NK cell dose within 48 hours of admission to the hospital for COVID. In yet another example embodiment, allogeneic, expanded NK cells are dosed by patient weight to quantitatively and qualitatively restore innate immune function against COVID. In this example embodiment, a dose of 107 NK cells/kg patient weight is provided to the COVID patient (e.g., a dose expected to replace the complete NK cell content of the peripheral blood in the average patient). In another example embodiment, COVID patients will receive up to 2 doses.

In another aspect, Source Peripheral Blood Mononuclear Cells (PBMCs) are collected and NK cells propagated as per standard methods. In yet another aspect, PBMC are depleted of CD3+ T cells using MACS colloidal super-paramagnetic CD3 MicroBeads. The resulting cells are co-cultured with irradiated feeder cells and/or membrane particles in media supplemented with fetal calf serum and IL-2. At Day 7, the cultures are the restimulated. In one aspect, the NK cell product undergo lot release testing and cryopreservation on day 14 for subsequent infusion. NK cells can be cryopreserved in single-dose aliquots (e.g., 50 mL containing 108 NK cells/mL). Assuming an initial donor blood draw equivalent to 1 unit (450 mL), a median content of 1.26×105 NK cells/mL, and a median expansion of 2,800-fold in 2 weeks, each donor can generate sufficient NK cells for 31 unit-dose bags. Assuming an initial donor apheresis containing a median of 3×108 NK cells after CD3 depletion (MD Anderson experience), each donor can generate an average of 168 unit-dose bags. One bag is sufficient for one dose of 108 NK cells/kg for a 50 kg individual. Doses of 108/kg can require up to 2-3 bags per patient per dose for adult patients. One example assumption is that freezing media contains 10% DMSO, the DMSO administered for a 108/kg dose will be 0.1 ml/kg.

VI. Genotyping, Sequencing, and Polymerase Chain Reaction (PCR) Immunoassays and Fluorochromes

The steps of various useful immunodetection methods have been described in the scientific literature. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Determination of expression levels of nucleic acid molecules in the practice of the inventive methods may be performed by any method, including, but not limited to, Southern analysis, Northern analysis, polymerase chain reaction (PCR) (see, for example, “PCR Protocols: A Guide to Methods and Applications”, Innis et al. (Eds.), 1990, Academic Press: New York), reverse transcriptase PCR(RT-PCT), anchored PCR, competitive PCR (see, for example, U.S. Pat. No. 5,747,251), rapid amplification of cDNA ends (RACE) (see, for example, “Gene Cloning and Analysis: Current Innovations, 1997, pp. 99-115); ligase chain reaction (LCR) (see, for example, EP 01 320308), one-sided PCR (Ohara et al., Proc. Natl. Acad. Sci., 1989, 86: 5673-5677), in situ hybridization, Taqman based assays (Holland et al., Proc. Natl. Acad. Sci., 1991, 88:7276-7280), differential display (see, for example, Liang et al., Nucl. Acid. Res., 1993, 21: 3269-3275) and other RNA fingerprinting techniques, nucleic acid sequence based amplification (NASBA) and other transcription based amplification systems, Qbeta Replicase, Strand Displacement Amplification (SDA), Repair Chain Reaction (RCR), nuclease protection assays, subtraction-based methods, Rapid-Scan™, and the like

Nucleic acid probes may be used in hybridization techniques to detect polynucleotides encoding for specific features of the NK cells. The technique generally involves contacting an incubating nucleic acid molecules in a biological sample obtained from a subject with the nucleic acid probes under conditions such that specific hybridization takes place between the nucleic acid probes and the complementary sequences in the nucleic acid molecules. After incubation, the non-hybridized nucleic acids are removed, and the presence and amount of nucleic acids that have hybridized to the probes are detected and quantified. Genotyping is performed through one of PCR, hybridization probes, and/or direct DNA sequencing.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label includes a fluorescent dye, a member of a binding pair, such as biotin/streptavidin, a metal (e.g., gold), and/or an epitope tag that specifically interacts with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they are detectable at very low amounts. Furthermore, in the case where multiple antigens are reacted with a single array, each antigen is labelable with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (5-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red; Acridine Yellow; Acriflavin; Acriflavin Feulgen SITSA; Aequorin (Photoprotein); AFPs—AutoFluorescent Protein-(Quantum Biotechnologies) see sgGFP, sgBFP; Alexa Fluor 350™; Alexa Fluor430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; B0130TM-1; BOBO™-3; Bodipy492/515; Bodipy493/503; Bodipy500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy F1; Bodipy FL ATP; Bodipy F1-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue; Calcium Crimson-; Calcium Green; Calcium Green-1 Ca2+ Dye; Calcium Green-2 Ca2+; Calcium Green-5N Ca2+; Calcium Green-C18 Ca2+; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA; Coelenterazine; Coelenterazine cp; Coelenterazine f; Coelenterazine fcp; Coelenterazine h; Coelenterazine hcp; Coelenterazine ip; Coelenterazine n; Coelenterazine 0; Coumarin Phalloidin; C-phycocyanine; CPM I Methylcoumarin; CTC; CTC Formazan; Cy2TM; Cy3.1 8; Cy3.5TM; Cy3TM; Cy5.1 8; Cy5.5 TM; Cy5 TM; Cy7 TM; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DiC18(5)); DIDS; Dihydorhodamine 123 (DHR); Dil (DilC18(3)); I Dinitrophenol; DiO (DiOC18(3)); DiR; DiR (Di1C18(7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 1OGF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342; Hoechst 34580; I IPTS; Hydroxycoumarin; Hydroxystilbamidine (Fluor® Gold); Hydroxytryptamine; Indo-1, high calcium; Indo-1 low calcium; Indodicarbocyanine (DiD); Indotricarbocyanine (DiR); Intrawhite Cf; JC-1; JO JO-1; JO-PRO-1; LaserPro; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-lndo-1; Magnesium Green; Magnesium Orange; Malachite Green; Marina Blue; I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feuigen); PBFI; PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 (Sigma); PKH67; PMIA; Pontochrome Blue Black; POPO-1; POPO-3; PO-PRO-1; PO-I PRO-3; Primuline; Procion Yellow; Propidium lodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARFI; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; Tetracycline; Tetramethylrhodamine (TRITC); Texas Red™; Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which are activatable with light or other electromagnetic energy source), or a combination thereof.

In one aspect, a modifier unit such as a radionuclide is incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide is attached to a linking group or bound by a chelating group, which is then attached to the compound directly or by means of a linker. Examples of radionuclides useful in this embodiment include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled described herein are conveniently usable in conjunction with imaging techniques such as positron emission tomography (PET) or single photon emission computerized tomography (SPECT).

Labeling is either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, produces a visible or detectable product at the site of the immunocomplex. ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule has a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avidin pair. In this mode, the detecting antibody or detecting molecule should include the other member of the pair.

Other modes of indirect labeling include the detection of primary immune complexes by a two-step approach. For example, a molecule (e.g., a first binding agent), such as an antibody, that has binding affinity for the molecule of interest or corresponding antibody can be used to form secondary immune complexes, as described above.

After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (e.g., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, e.g., where the substance can be found in a subject, tissue or cell.

Provided that the concentrations are sufficient, the molecular complexes ([Ab-Ag]n) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field. Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices—agarose and polyacrylamide—provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucicotides that require a small gel pore size for retardation.

Proteins are amphoteric compounds; their net charge therefore is determined by the pI of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids arc linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone—and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTI). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log l OMW for known samples, and read off the log Mr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection.

Generally, proteins are separated by gel electrophoresis, usually SDS-PAGE. The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g., alkaline phosphatase or horseradish peroxidase) or chemiluminescent substrates. Other possibilities for probing include the use of fluorescent or radioisotope labels (e.g., fluorescein, 1251). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) are usable to detect the interactions between DNA binding proteins and their cognate DNA recognition sequences, in both a qualitative and quantitative manner.

In a general gel-shift assay, purified proteins or crude cell extracts can be incubated with a labeled (e.g., 32P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Gel shift methods can include using, for example, colloidal forms of COOMASSIE (Imperial Chemicals Industries, Ltd) blue stain to detect proteins in gels such as polyacrylamide electrophoresis gels. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis. Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, and/or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and/or magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes 1251 or 1311 are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, B-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are known to those of skill in the art. In one variation, antibodies that bind to proteins are immobilized onto a selected surface exhibiting protein affinity, such as a well in a polystyrene microtiter plate. Then, a test composition suspected of containing a marker antigen is added to the wells. After binding and washing to remove non-specifically bound immunocomplexes, the bound antigen are detectable.

Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal. Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISPOT) is an immunoassay that can detect an antibody specific to a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, B-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C. Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting method, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and 1-1202, in the case of peroxidase as the enzyme label. Quantitation is then achievable by measuring the degree of color generation, e.g., using a visible spectra spectrophotometer. Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment. Bioinformatics support is important; the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, e.g., protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods are automatable for high throughput production. For capture arrays and protein function analysis, it is important that proteins should be correctly folded and functional; this is not always the case, e.g., where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, N.J.) and specialized chip designs, such as engineered microchannels in a plate (e.g., The Living Chip™, Biotrove, Woburn, Mass.) and tiny 3D posts on a silicon surface (Zyomyx, Hayward Calif.). Particles in suspension can also be used as the basis of arrays, providing they are coded for identification; systems include colour coding for microbeads (Luminex, Austin, Tex.; Bio-Rad Laboratories) and semiconductor nanocrystals (e.g., QDots™, Quantum Dot, I layward, CA), and barcoding for beads (UltraPlex™, SmartBead Technologies Ltd, Babraham, Cambridge, UK) and multimetal microrods (e.g., Nanobarcodes™ particles, Nanoplex Technologies, Mountain View, Calif.). Beads can also be assembled into planar arrays on semiconductor chips (LEAPS technology, BioArray Solutions, Warren, N.J.).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (such as, for example, size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the VersalinxrM system (Prolinx, Bothell, Wash.) reversible covalent coupling is achieved by interaction between the protein derivatised with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, Mass.), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilized on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g., produced and sold by Packard Biosciences] as well as manual equipment [e.g., produced and sold by V & P Scientific]. Bacterial colonies can be robotically gridded onto PVDF membranes for induction of protein expression in situ. At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g., Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (e.g., produced and sold by FITS Biosystems, Intrinsic Bioprobes, Tempe, Ariz.), rolling circle DNA amplification (e.g., produced and sold by Molecular Staging, New Haven Conn.), mass spectrometry (e.g., produced and sold by Intrinsic Bioprobes; Ciphergen, Fremont, Calif.), resonance light scattering (e.g., produced and sold by Genicon Sciences, San Diego, Calif.) and atomic force microscopy (e.g., produced and sold by BioForce Laboratories).

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner. Antibody arrays have the required properties of specificity and acceptable background, and some are available commercially (e.g., as produced and sold by BD Biosciences, San Jose, Calif.; Clontech, Mountain View, Calif.; and/or BioRad; Sigma, St. Louis, Mo.). Antibodies for capture arrays are made either by conventional immunization (polyclonal sera and hybridomas), or as recombinant fragments, usually expressed in E. coli, after selection from phage or ribosome display libraries (e.g., produced and sold by Cambridge Antibody Technology, Cambridge, UK; Biolnvent, Lund, Sweden; Affitech, Walnut Creek, Calif.; and/or Biosite, San Diego, Calif.). In addition to the conventional antibodies, Fab and scFv fragments, single V-domains from camelids or engineered human equivalents (e.g., produced and sold by Domantis, Waltham, Mass.) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (e.g., produced and sold by Affibody, Bromma, Sweden), ‘Trinectins’ based on fibronectins (e.g., produced and sold by Phylos, Lexington, Mass.) and ‘Anticalins’ based on the lipocalin structure (e.g., produced and sold by Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (e.g., produced and sold by SomaLogic, Boulder, Colo.). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements.

Aptamers have the advantages of ease of production by automated oligonucleotide synthesis and the stability and robustness of DNA; on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix; the cavities can then specifically capture (denatured) proteins that have the appropriate primary amino acid sequence (e.g., produced and sold as ProteinPrint™, by Aspira Biosystems, Burlingame, Calif.).

Another methodology which can be used diagnostically and in expression profiling is the ProteinChip® array (e.g., produced and sold by Ciphergen, Fremont, Calif.), in which solid phase chromatographic surfaces bind proteins with similar characteristics of charge or hydrophobicity from mixtures such as plasma or tumor extracts, and SELDI-TOF mass spectrometry is used to detection the retained proteins. Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g., via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilised on a microarray. Large-scale ‘proteome chips’ promise to be very useful in identification of functional interactions, drug screening, etc. (e.g., produced and sold by Proteometrix, Branford, Conn.).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, e.g., through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

V. Method of Using the Composition

In one aspect, disclosed herein are methods of treating, preventing, inhibiting, and/or reducing a cancer, metastasis, or an infectious disease in a subject comprising administering to the subject any of the isolated or engineered universal donor NK cell or cell line disclosed herein or any universal donor NK cell or cell line or engineered universal donor NK cell or cell line that is selected by or screened by the methods 300, 400 or prepared by any of the methods disclosed herein.

For example, in one aspect, disclosed herein are methods of treating a cancer or an infectious disease in a subject comprising identifying and/or obtaining universal donor cells as described in method 300 of FIG. 3, and/or engineering universal donor cells as described in method 400 of FIG. 4. In another aspect, the method of treating a cancer or an infectious disease in a subject comprising identifying and/or obtaining universal donor cells comprises (a) obtaining or having obtained a HLA genotype of candidate NK cells from an NK cell donor, wherein the HLA genotype is indicative of the presence or absence of HLA CI, C2, and Bw4 alleles and thereby indicative of the presence of one or more variably inherited inhibitory KIRs 2DL1, 2DL2, 2DL3, and 3DL1; (b) obtaining or having obtained a KIR genotype of the candidate NK cells, wherein the KIR genotype is indicative of the presence or absence of activating KIRs selected from the group consisting of 2DS1/2, 2DS3/5, 3DS1, and 2DS4; and (c) selecting the candidate NK cells as a universal donor NK cell for the therapeutic administration when (i) the HLA genotype indicates the presence of at least two I ILA alleles HLA CI, C2, and Bw4; and (ii) the KIR genotype indicates the presence of at least three activating KIRs 2DS1/2, 2DS3/5, 3DS I, and/or 2DS4.

In one aspect, disclosed herein are methods treating a cancer or an infectious disease, wherein the selected universal donor NK cells are histologically optimized with at least 50%-85% of recipient subjects. In one aspect the methods of treating a cancer or an infectious disease of any preceding aspect, further comprising obtaining or having obtained the CMV seropositivity of the candidate NK cells; and wherein the NK candidate NK cells are further selected when the NK cell donor is seropositive for CMV or the NK cells from the NK cell donor have high NKG2C expression compared to a reference level of NKG2C expression.

Method 500 illustrated in FIG. 5, and continued from above, recites methods of treatment for a particular patient starting at 520. At step 520, the NK cells are generated at a concentration within a percentage of an assigned dose level of a patient/recipient. In one example embodiment, the concentration of TGF-βi NK cells/kg is within 20% of a patient's assigned dose level. For each patient to be treated with the NK cells, a platelet-reactive antibody test is performed to allow exclusion of TGF-βi NK cell products from donors with HLA types to which the patient has been allo-immunized. The patient's body weight is used for calculation of TGF-βi NK dose, the patient's assigned dose level, and planned infusion dates. In one example embodiment, stored TGF-βi NK cells from remaining donors (e.g., donors that were not excluded) are prepared for distribution for each dose. The doses are verified for NK cell, T cell, and endotoxin doses.

At step 522, the CD3+ cells present in the NK Cells are determined to be below a T-cell threshold of the assigned dose level. If CD3+ cells present in the NK Cells are determined to be above a T-cell threshold of the assigned dose level, the dose is excluded. In one example embodiment, the T-cell threshold is less than or equal to the maximum cumulative T-Cell does (see Table 2, below) of the patient's assigned dose level.

At step 524, the endotoxin dose of the non-excluded donor cells is determined to be less than or equal to an endotoxin threshold and identified as donor eligible cells. In one example embodiment, the endotoxin threshold is less than or equal to 5 EU/kg. At step 526, doses of the NK cells are provided to the patient for a threshold dose cycle. In one example embodiment, the threshold dose cycle is 6 cycles of 21 days each consisting of irinotecan, temozolomide, dinutuximab, and sargramostim, and universal donor TGF-βi ex vivo expanded NK cells (e.g., the donor eligible cells). The Universal Donor, expanded, TGF-βi NK cells are administered by IV on day 8 of the 21 day cycle at a dose of 1×10⁸ NK cells/kg patient weight. In one example embodiment, there is dose escalation. In another example embodiment, there is no dose escalation.

It is understood and herein contemplated that activating and/or expanding the universal donor NK cells prior to therapeutic administration to a subject can help overcome many hurdles associated with cytokine toxicity. In one aspect, the methods treating a cancer or an infectious disease of any preceding aspect, further comprising incubating the selected universal donor NK cells in vitro in the presence of one or more NK cell effector agents (e.g., stimulatory peptides, cytokines, and/or adhesion molecules) (for example IL-21). Examples of NK cell activating agents and stimulatory peptides include, but are not limited to IL-21, 41BBL, IL-2, IL-12, IL-15, IL-18, IL-7, ULBP, MICA, LFA-1, 2B4, BCM/SLAMF2, CCR7, OX4OL, NKG2D agonists, Delta-1, Notch ligands, NKp46 agonists, NKp44 agonists, NKp30 agonists, other NCR agonists, CD16 agonists; and/or TGF-β and/or other homing inducing signaling molecules. Examples of cytokines include, but are not limited to, IL-2, IL-12, IL-21, and IL-18. Examples of adhesion molecules include, but are not limited to LFA-1, MICA, BCM/SLAMF2.

These NK cell effector agents are soluble presented in solution or present as membrane bound agent on the surface of plasma membrane (PM) particles, exosome (EX), or feeder cells (FC). The PM particles, EX exosomes, and/or FC cells can be engineered to express membrane forms of the NK cell activating agents and stimulatory peptides. Alternatively, the NK cell activating agents and stimulatory peptides can be chemically conjugated to the surface of the PM particle, EX exosome, of FC feeder cell. For example, a plasma membrane (PM) particle, Feeder cells (FC), or exosomes (EX) prepared from feeder cells expressing membrane bound IL-21 (FC21 cells, PM21 particles, and EX21 exosomcs, respectively). It is understood and herein contemplated that the membrane bound IL-21 expressing FC21 cells, PM21 particles, and EX21 exosomes can further comprise additional one or more activating agents, stimulatory peptides, cytokines, and/or adhesion molecules including, but not limited to 41BBL, IL-2, IL-12, IL-15, IL-18, IL-7, ULBP, MICA, LEA-I, 2B4, BCM/SLAMF2, CCR7, OX4OL, NKG2D agonists, Delta-1, Notch ligands, NKp46 agonists, NKp44 agonists, NKp30 agonists, other NCR agonists, CD16 agonists; and/or TGF-β (for example, PM21 particle, EX21 exosome, or FC cell expressing 41BBL and membrane bound interleukin-21).

It is understood that the pathogen can be a virus. Thus in one embodiment the pathogen can be selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, I lepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and/or Human Immunodeficiency virus type-2.

Also disclosed are methods wherein the pathogen is a bacterium. The pathogen can be selected from the group of bacteria consisting of Mycobaterium tuberculosis, Mycobaterium bovis, Mycobaterium bovis strain BCG, BCG substrains, Mycobaterium avium, Mycobaterium intracellular, Mycobaterium africanum, Mycobaterium kansasii, Mycobaterium marinum, Mycobaterium ulcerans, Mycobaterium avium subspecies paratuberculosis, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsia! species, Ehrlichia species, Staphylococcus aureus, Staphylococcus epidermidis, Streptococcus pneumoniac, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and/or other Yersinia species, and/or Mycoplasma species. In one aspect the bacteria is not Bacillus anthracis.

Also disclosed are methods of treating an infectious disease wherein the pathogen is a fungus selected from the group of fungi consisting of Candida albicans, Cryptococcus neoformans, Histoplama capsulatum, Aspergillus fumigates, Coccidiodes immitis, Paracoccidioides brasiliensis, Blastomyces dermitidis, Pneumocystis carinii, Penicillium marneffi, and/or Alternaria alternata.

Also disclosed are methods of treating an infectious disease wherein the pathogen is a parasite selected from the group of parasitic organisms consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Trichobilharzia regenti, Trichinella spiralis, Trichinella britovi, Trichinella nelsoni, Trichinella nativa, and/or Entamoeba histolytica.

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, thyroid cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, stomach cancer, prostatic cancer, and/or pancreatic cancer.

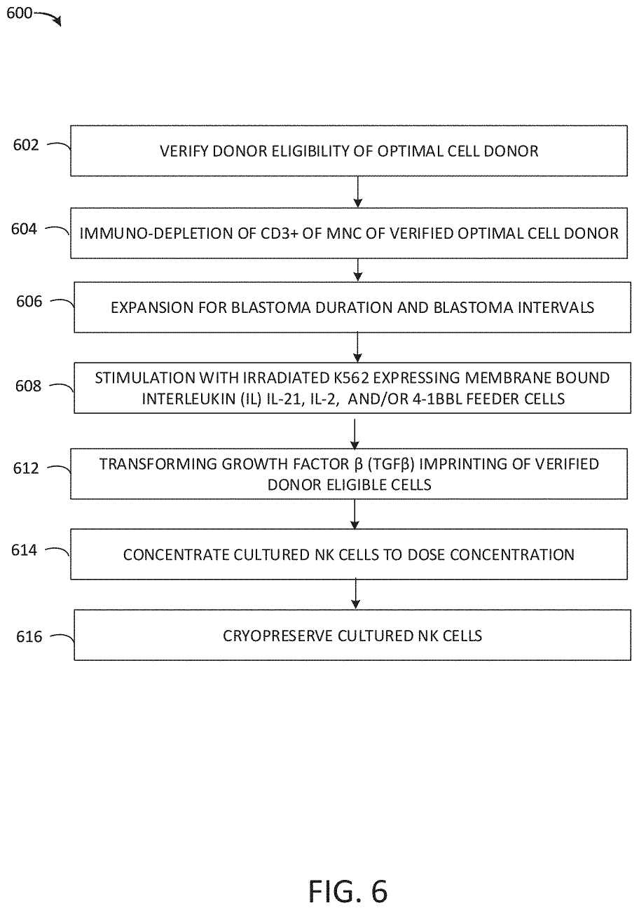

In the illustrated example embodiment FIG. 6, the NK cells are utilized in treatment preparation method 600 to treat cancers, such as neuroblastoma. At step 602, donor eligibility as an optimal donor is verified. In one example embodiment, the optimal donor is verified as described above in FIG. 3, and/or optimal donor cells are engineered as in FIG. 4. Thus the optimal donor is one who has an HLA genotype carrying C1, C2, and Bw4 alleles, has a KIR genotype possessing the inhibitory KIR (2DL1, 2DL2 or 3, and 3DL1) that bind to C1, C2, and Bw4 (leading to maximum licensing) and with a high proportion of activating KIR (greater than or equal to 3 of the variably-inherited activating genes including 2DS1 and 3DS1), and has been exposed to CMV resulting in high NKG2C expression.

At step 604, the CD3+ immune-depletion of MNCs of optimal cell donors is performed. In one example embodiment, the CD3+ immune-depletion is the same as in step 506 of method 500. At step 606, the depleted optimal donor cells are expanded for a blastoma duration for blastoma intervals. In one example embodiment, the blastoma duration is between 10-18 days. In another example embodiment, the blastoma duration is 14 days. In another example embodiment, the blastoma intervals (e.g., when expansion inducing elements are added) is 1-3 days. During expansion, at step 608, the depleted optimal donor cells are stimulated with irradiated k562 expressing membrane bound interleukin (Il) Il-21, Il-2, and/or 4-1BBL feeder cells. In one example embodiment, NK cells are generated during the stimulation using the irradiated K562 expressing membrane bound IL-21 and 4-1BBL as well as IL-2 (e.g., at concentration 100 IU/mL) feeder cells. The irradiated feeder cells (IFCs) are added at an approximate 1:2 TNC-to-IFC ratio in the first seven days of the blastoma duration and 1:1 ratio in the second seven days of the blastoma duration. In one example embodiment, fresh IL-2 is added every blastoma interval.

At 612, transforming growth factor β (TGF-β) is used or imprinted on verified donor eligible cells to generate TGF-βi NK cells. In one example embodiment, the donor eligible cells are chronically stimulated by TGF-β (e.g., at concentration 10 ng/mL). In another example embodiment, fresh TGF-β is added every blastoma interval during the blastoma duration. The addition of TGF-β during the expansion process impairs neither fold expansion (465-3200-fold expansion) nor viability (>96%) of the final expanded NK cell product. TGF-βi NK cells exhibit a pro-inflammatory phenotype with hypersecretion of interferon-gamma and tumor necrosis factor-alpha when cultured with tumor targets, which increased anti-tumor cytokine secretion owing both to the increased percentage of cytokine-producing NK cells in culture and to the amount of cytokine each of these cells produce compared to typically expanded NK cells. These cells have phenotypic and transcriptional changes that confer resistance to suppression by TGF-β.

At 614, the cultured NK cells are concentrated into a dose concentration. In one example, the dose concentration is between 2×10⁶ NC/mL and 2×10⁸ NC/mL. At 616, the expanded and transformed NK cells at the dose concentration are cryopreserved. In one embodiment, the NK cells are cryopreserved in NK Freeze Media. In another example embodiment, the NK Freeze Media comprises 10% DMSO, 12.5% (w/v) human serum albumin (HSA), USP, and/or In Plasma-Lyte A (USP).

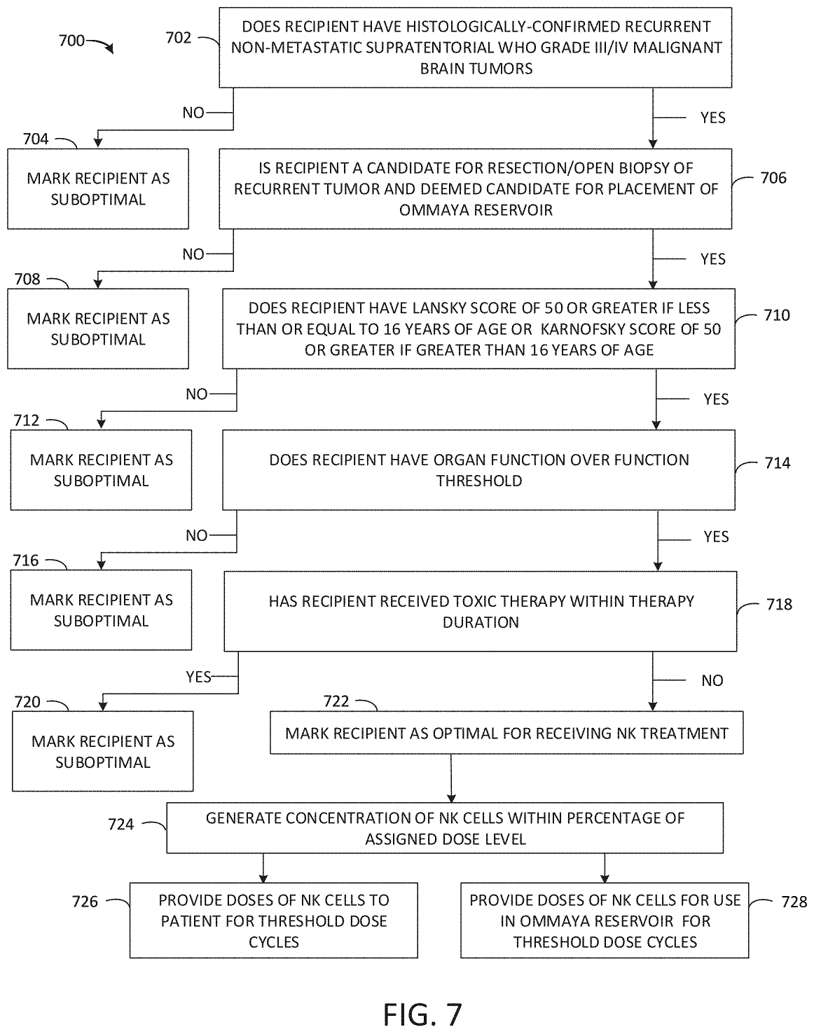

In the illustrated example embodiment FIG. 7, recipient/patient eligibility and treatment with the NK cells are described in recipient eligibility and treatment method 700 to treat one or more cancers, such as neuroblastoma. At 702, it is determined if the recipient has histologically-confirmed recurrent non-metastatic supratentorial World Health Organization (WHO) grade III/IV malignant brain tumors. In one example embodiment, the brain tumor includes anaplastic ependymoma, embryonal tumor, primitive neuroectodermal tumor, AT/RT, anaplastic astrocytoma, anaplastic oligoastrocytoma, anaplastic oligodendroglioma, anaplastic pleomorphic xanthoastrocytoma, glioblastoma multiforme, gliosarcoma, and/or malignant glioma NOS.

At 704, responsive to the recipient lacking histologically-confirmed recurrent non-metastatic supratentorial WHO grade III/IV malignant brain tumors, the recipient is marked as sub-optimal (e.g., not a candidate for receiving NK donor cells). At 706, responsive to the recipient having histologically-confirmed recurrent non-metastatic supratentorial WHO grade III/IV malignant brain tumors, it is determined if recipients are candidates for resection/open biopsy of the recurrent tumor (resection candidates) and/or be deemed candidates for placement of an Ommaya reservoir placed intra-cavitary/intra-tumoral (Ommaya candidate). At 708, responsive to the recipient not being deemed a resection candidate or an Ommaya candidate, the recipient is marked as sub-optimal.

At 710, responsive to the recipient being deemed a resection candidate and/or an Ommaya candidate, it is determined if the recipient has a Lansky score of 50 or greater if the recipient is less than or equal to 16 years of age (optimal Lansky score) or a Karnofsky score of 50 or greater if the recipient is over 16 years of age (optimal Karnofsky score). In one example embodiment, optimal candidates are greater than or equal to 3 years of age and less than 25 years of age at the time of entry into the study. At 712, responsive to the recipient not being deemed to have optimal Lansky score or optimal Karnofsky score for their age, the recipient is marked as sub-optimal.

At 714, responsive to the recipient being deemed to have optimal Lansky score or optimal Karnofsky score for their age, it is determined if the recipient has organ function over a function threshold. In one example embodiment, the function threshold is having adequate bone marrow function, without transfusion or growth factors within 21 days of NK cell administration. In another example embodiment, adequate bone marrow function is defined as a white blood cell (WBC) greater than or equal to 2.5×103/microliter, hemoglobin (Hgb) greater than or equal to 9 gm/dL, absolute neutrophil count (ANC) greater than or equal to 1,000 cells/microliter and platelet count of greater than or equal to 75,000 cells/microliter. In one example embodiment, the function threshold is having adequate liver function and/or adequate renal function. In one example embodiment, adequate liver function is defined wherein ALT, AST and alkaline phosphatase is less than 2 times ULN, and bilirubin less than 1.5 times ULN, and adequate renal function is defined wherein BUN or creatinine less than 1.5 times ULN. At 716, responsive to the recipient not being deemed to have organ function over the organ function threshold, the recipient is marked as sub-optimal.

At 718, responsive to the recipient being deemed to have organ function over the organ function threshold, it is determined whether the recipient has received toxic therapy within the therapy duration. In one example embodiment, optimal recipients have completed first-line treatment with radiation and/or chemotherapy prior to receiving universal donor NK cell treatment. In one example embodiment, the therapy duration is at least 12 weeks since the completion of initial radiation therapy. In another example embodiment, the therapy duration is at least 6 weeks since the completion of any cytotoxic chemotherapy regimen. In yet another example embodiment, the therapy duration a minimum of 2 weeks since the last dose of any toxic agent. In this example embodiment, the recipient is deemed to have recovered from any toxicity of the toxic agent prior to treatment of the universal NK donor cells. In one example embodiment, the therapy duration is between diagnosis of cancer and a current time. In another example embodiment, the toxic therapy is systemic steroids (except replacement therapy), and the therapy duration is at least 3 days prior to NK cell infusion. In another example embodiment, the toxic therapy is bevacizumab, and the therapy duration is at least 6 weeks before starting NK cell infusion. At 720, responsive to the recipient being deemed to have received toxic therapy within the therapy duration, the recipient is marked as sub-optimal. At 722, responsive to the recipient being deemed to have received toxic therapy outside the therapy duration, the recipient is marked as optimal for receiving universal donor NK cell therapy.

At 724, NK cells (generated using method 600 of FIG. 6) are generated having the concentration of NK cells within a percentage of an assigned dose level (e.g., as recited in Table 2, below). In one example embodiment, the duration of therapy is 3 months and/or until disease progression, inter-current illness that prevents further administration of treatment, unacceptable adverse event(s), patient decides to withdraw, significant patient non-compliance with protocol, general or specific changes in the patient's condition render the patient unacceptable for further treatment in the judgment of the clinician. At 726, doses of NK cells are provided for use in the optimal recipient for the threshold dose cycle (e.g., see Table 2, below). In one example, the doses of NK cells are provided through intravenous, intramuscular, etc. methods.

At 728, doses of NK cells are provided for use in an Ommaya reservoir for the threshold dose cycle (e.g., see Table 2, below). Patients proceed to surgery for tumor resection and Ommaya placement. In one example embodiment, a first dose of TGFβi NK cells is administered at least 14 days after the Ommaya reservoir placement. TGFβi NK cell infusions through the Ommaya reservoir will occur once weekly for three weeks followed by one rest week for a total of three (four week) cycles. If patients have stable or improved disease, then patients continue to receive therapy for a total of 12 cycles. In one example embodiment, the optimal recipient receives 3 cycles of TGFβi NK cell infusion. Each cycle is of 4 weeks duration. During the first 3 weeks, TGFβi NK cells are infused once weekly. The 4th week is a rest week. TGFβi NK cell infusions should be delivered at least 3 days apart (e.g., Friday of Week 1 and Monday of Week 2). Dosing is based on recipient body surface area (BSA).

TABLE 2
Dose Levels and Cumulative Amounts

| | NK cell | Cumulative NK | Cumulative NK | Maximum |
|---|---|---|---|---|
| Dose | number per | cell number per | cell number per | cumulative T |
| Level | infusion | cycle | dose level | cell dose |
| 0 | 3 × 10⁵/m² | 9 × 10⁵/m² | 2.7 × 10⁶/m² | 8.9 × 10³/m² |
| 1 | 3 × 10⁶/m² | 9 × 10⁶/m² | 2.7 × 10⁷/m² | 8.9 × 10⁴/m² |
| (starting | | | | |
| dose) | | | | |
| 2 | 3 × 10⁷/m² | 9 × 10⁷/m² | 2.7 × 10⁸/m² | 8.9 × 10⁵/m² |
| 3 | 3 × 10⁸/m² | 9 × 10⁸/m² | 2.7 × 10⁹/m² | 8.9 × 10⁶/m² |

Week 4: Rest week

It is also contemplated herein that the disclosed methods of treating, preventing, inhibiting, or reducing a cancer or metastasis in a subject can further comprise the administration of any anti-cancer agent that would further aid in the reduction, inhibition, treatment, and/or elimination of the cancer or metastasis (such as, for example, gemcitabine). Anti-cancer agents that can be used in the disclosed bioresponsive hydrogels or as an additional therapeutic agent in addition to the disclosed pharmaceutical compositions, engineered particles, and/or bioresponsive hydrogels (including bioresponsive hydrogels that have an engineered particle encapsulated therein) for the methods of reducing, inhibiting, treating, and/or eliminating a cancer or metastasis in a subject disclosed herein can comprise any anti-cancer agent known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Am inolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubr is (Carmustine), Carmustine, Carmustine Implant, Casodex (Bicalutamide), CEM, Ceritinib, Cerubidine (Daunorubicin I lydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasonc, Dexrazoxane I lydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (V ismodegib), Erlotinib I lydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gcmtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Ilydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), lnotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Pali fermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant I luman Papillomavirus (1-IPV) Bivalent Vaccine, Recombinant I luman Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan I lycela (Rituximab and I Iyaluronidase Human), Rituximab, Rituximab and, 1-lyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Zytiga (Abiraterone Acetate). Checkpoint inhibitors include, but are not limited to antibodies that block PD-1 (Nivolumab (BMS-936558 or MDX1106), CT-011, MK-3475), PD-LI (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

### REFERENCES

Almalte Z, Samarani S, Iannello A, et al. Novel associations between activating killer-cell immunoglobulin-like receptor genes and childhood leukemia. Blood. 2011; 118(5):1323-1328.
Braud V M, Allan D S, O'Callaghan Calif., et al. HLA-E binds to natural killer cell receptors CD94/NKG2A, B and C. Nature. 1998; 391(6669):795-799.
Cichocki F, Cooley S, Davis Z, et al. CD56dimCD57+NKG2C+NK cell expansion is associated with reduced leukemia relapse after reduced intensity HCT. Leukemia. 2016; 30(2):456-463.
Foley B, Cooley S, Verneris M R, et al. Cytomegalovirus reactivation after allogeneic transplantation promotes a lasting increase in educated NKG2C+ natural killer cells with potent function. Blood. 2012; 119(11):2665-2674.
Foley B, Cooley S, Verneris M R, et al. Human cytomegalovirus (CMV)-induced memory-like NKG2C(+) NK cells are transplantable and expand in vivo in response to recipient CMV antigen. J Immunol. 2012; 189(10):5082-5088.
Mancusi A, Ruggeri L, Urbani E, et al. Haploidentical hematopoietic transplantation from KIR ligand-mismatched donors with activating KIRs reduces nonrelapse mortality. Blood. 2015; 125(20):3173-3182.
Pittari G, Fregni G, Roguet L, et al. Early evaluation of natural killer activity in post-transplant acute myeloid leukemia patients. Bone Marrow Transplant. 2010; 45(5):862-871.
Ruggeri L, Mancusi A, Perruccio K, Burchielli E, Martelli MF, Velardi A. Natural killer cell alloreactivity for leukemia therapy. J Immunother. 2005; 28(3):175-182.
Stringaris K, Adams S, Uribe M, et al. Donor KIR Genes 2DL5A, 2DS1 and 3DS1 are associated with a reduced rate of leukemia relapse after HLA-identical sibling stem cell transplantation for acute myeloid leukemia but not other hematologic malignancies. Biol Blood Marrow Transplant. 2010; 16(9):1257-1264.

In the foregoing specification, specific embodiments have been described. however, one of ordinary skill in the art appreciates that various modifications and changes can be made without departing from the scope of the disclosure as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The disclosure is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art. In one non-limiting embodiment the terms are defined to be within for example 10%, in another possible embodiment within 5%, in another possible embodiment within 1%, and in another possible embodiment within 0.5%. The term “coupled” as used herein is defined as connected or in contact either temporarily or permanently, although not necessarily directly and not necessarily mechanically. A device or structure that is “configured” in a certain way is configured in at least that way, but may also be configured in ways that are not listed.

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting. The references disclosed are also individually and specifically incorporated by reference herein for the material contained in them that is discussed in the sentence in which the reference is relied upon.

To the extent that the materials for any of the foregoing embodiments or components thereof are not specified, it is to be appreciated that suitable materials would be known by one of ordinary skill in the art for the intended purposes. Any item, text, patent, patent publication, patent application no. referenced herein is incorporated herein by reference in their entireties for all purposes.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points.

For example, if a particular data point “20” and a particular data point 25 are disclosed, it is understood that greater than, greater than or equal to, less than, less than or equal to, and equal to 20 and 25 are considered disclosed as well as between 20 and 25. It is also understood that each unit between two particular units are also disclosed. For example, if 20 and 25 are disclosed, then 21, 22, 23, and 24 are also disclosed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

### EXAMPLES

In order that the invention may be more fully understood, the following examples are set forth. The examples described in this application are offered to illustrate the methods and compositions provided herein and are not to be construed in any way as limiting their scope.

### Example 1: Selecting the “Ideal” Donors to Generate Consistent and Potent “Off-the-Shelf” NK Cell Therapeutic Products

NK cells are licensed (acquire enhanced killing ability) when they express inhibitory killer immunoglobulin receptors (KIR) for self-HLA class I molecules. This enables NK cells to recognize “self” and spare autologous cells from killing. Targets lacking self-HLA class I molecules are thus more likely to elicit recognition by licensed NK cells. The inhibitory KIR genes known to be relevant for NK alloreactivity are: (i) 2DL1 which binds to HLA-C group 2 alleles, (ii) 2DL2 and 2DL3 which bind to HLA-C group 1 alleles, (iii) and 3DL1 which binds to HLA-B Bw4 alleles. According to the missing-ligand model, for each NK cell expressing an inhibitory KIR gene there will be alloreactivity only if the corresponding ligand is absent in the recipient, and present in the donor, e.g., any donor possessing a Group C1 allele will be alloreactive to any individual lacking a Group C1 allele. Thus, donors who possess HLA in the C1, C2, and Bw4 families are predicted by this model to be alloreactive against any recipient lacking C1, or C2, or Bw4.

Whereas inhibitory KIRs prevent alloreactivity, activating KIRs recognize activating ligands that promote NK cell lysis. Inheritance of activating KIR is widely variable 0 to 7 aKIR are possible in any one individual. Data from patients undergoing stem cell transplantation show that patients receiving allografts from donors with more activating KIRs have a better outcome than patients receiving allograft from donors with fewer activating KIR. Others have shown a protective benefit against leukemia in individuals that inherit more activing KIRs. Our laboratory has shown that NK cells with higher numbers of activating KIR induce stronger lysis of target cells (FIG. 1). In addition, the activating KIR 2DS1 and 3DS1 are associated with disease-free survival in multivariate analysis.

Lastly, NKG2C is an activating receptor that is expressed late in NK cell development and recognizes HLA-E rather than -B or -C. NKG2C expression is induced in patients with CMV infection and correlates with an adaptive NK cell phenotype and improved leukemia-free survival.

Thus the “optimal” donor is one who has an HLA genotype carrying C1, C2, and Bw4 alleles, has a KIR genotype possessing the inhibitory KIR (2DL1, 2DL2 or 3, and 3DL1) that bind ton C1, C2, and Bw4 (leading to maximum licensing) and with a high proportion of activating KIR (>3 of the variably-inherited activating genes including 2DS1 and 3DS1), and has been exposed to CMV resulting in high NKG2C expression.

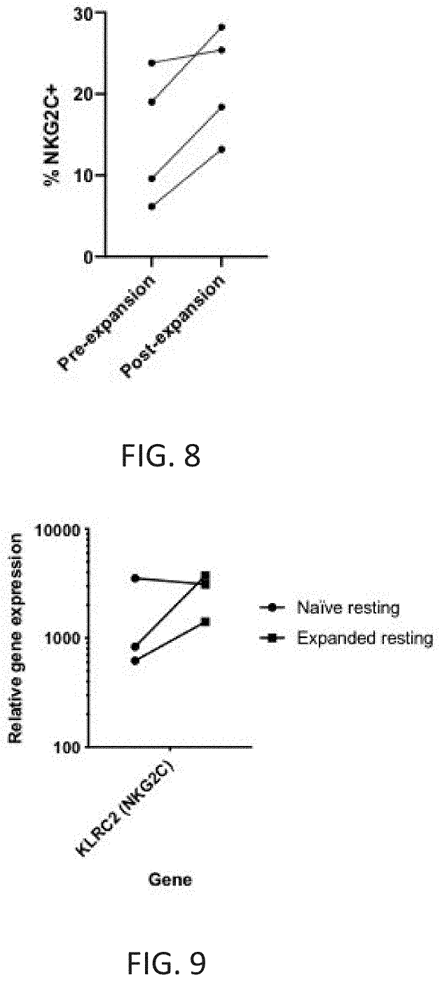

Considering data available for Caucasian donors, C1/C2/Bw4 alleles occur in 32% of the population. Of the 23 KIR genotypes that account for 80% of the population, 25.3% meet all of these criteria. ˜90% of adults will have been exposed to CMV. As illustrated in FIG. 8 by flow cytometry, all CMV+ donors have NK cells expressing NKG2C, and this is increased after expansion, such as described at 606 of the method 600 illustrated in FIG. 6. As illustrated in FIG. 9 by mRNA level measures, NKG2C expression increased after expansion, such as described at 606 of the method 600 illustrated in FIG. 6.

Thus, the “ideal” NK cell donor can be identified in approximately 1 out of 16 healthy individuals.

### Donor Selection:

For maximum cost savings and time efficiency, donors are screened in step-wise algorithm excluding donors from further testing who do not meet criteria.

Donor selection involves HLA and KIR genotyping, KIR phenotyping, and NK production (FIG. 5A, top). Donors may be KIR typed to assess the presence (grey) or absence (black) of KIR genes (FIG. 5A, bottom). In one example, PBMCs and donor matched NK-cells were analyzed by flow cytometry to determine KIR expression on NK cells. Expression of 2DL2/3, 2DL1 and 3DL1 was evaluated using KIR-specific antibodies REA147/CH-L,143211 and DX9, respectively. The percentage of NK cells expressing each KIR for individual donors was determined (FIG. 5B).

KIR genotyping may be performed for NK cell donors with reverse sequence-specific oligonucleotide (SSO) methodology (e.g., One Lambda), to enable discrimination of Functional vs. Deletion variants of KIR2DL4. KIR-B content can be determined using the B Content Calculator maintained by EMBL-EBI (www.ebi.ac.uk/ipd/kir/donor_b_content.html). Activating KIR content will be determined by scoring the total number of activating KIR genes. All DS-designated KIR and Functional KIR2DL4 are considered activating. Donors will be selected who have the common activating KIRs (KIR2DS4 and the functional version of KIR2DL4) and at least 3 of the 5 variably-inherited activating KIRs.

NK cell donors may be HLA typed at high-resolution level for alleles at HLA-B and C loci by SSO-PCR (amplification and oligonucleotide sequencing) using commercial kits. KIR-ligand class can be predicted using the KIR Ligand Calculator maintained by the European Bioinformatics Institute of the European Molecular Biology Labs (EMBL-EBI) (www.ebi.ac.uk/ipd/kir/ligand.html). Individuals possessing all three C1, C2, and Bw4 classes should be selected.

Donors are also tested for CMV. CMV+ donors are tested to confirm the presence of NKG2C+NK cells.

### Manufacturing and Vialing Estimates for OTS NK Cell Products:

The expanded donor NK cell product is manufactured prior to subject enrollment. All donors undergo standard infectious disease screening and other donor screening (as required by 21 CFR 1271 subpart C) within 7 days of collection. Source PBMCs are collected and NK cells propagated according to the procedures outlined in the CMC section of the FDA IND application. Briefly, PBMC are depleted of CD3+ T cells using MACS colloidal super-paramagnetic CD3 MicroBeads. The resulting cells are cocultured with irradiated feeder cells and/or membrane particles in media supplemented with fetal calf serum and IL-2. At Day 7, the cultures are re-stimulated. The NK cell product undergoes lot release testing and cryopreservation on day 14 for subsequent infusion. Sterility testing is partially completed at the time of cryopreservation, and all tests are final prior to release of the product. NK cells are cryopreserved in single-dose aliquots of 50 mL containing 10⁸ NK cells/mL. Assuming an initial donor blood draw equivalent to 1 unit (450 mL), a median content of 1.26×10⁵ NK cells/mL, and a median expansion of 2,800-fold in 2 weeks, each donor generates sufficient NK cells for 31 unit-dose bags. Assuming an initial donor apheresis containing a median of 3×10⁸ NK cells after CD3 depletion, each donor could generate an average of 168 unit-dose bags. One bag is sufficient for one dose of 10⁸ NK cells/kg for a 50 kg individual. Doses of 10⁸/kg may require up to 2-3 bags per patient per dose for adult patients. Assuming freezing media containing 10% DMSO, the DMSO administered for a 10⁸/kg dose will be 0.1 ml/kg.

### Example 2: Phase I/II Clinical Trial Testing the Safety and Feasibility of IL-21-Expanded Natural Killer Cells for the Induction of Relapsed/Refractory Acute Myeloid Leukemia

### 1.0 Background and Rationale

### 1.1 Relapsed AML and Hematopoietic Stem Cell Transplantation (HSCT)

Hematopoietic stem cell transplantation (HSCT) is an effective treatment for AML. With HSCT the long term disease free survival rate is approximately 60% for patients transplanted in first remission. After relapse the rate falls to approximately 40% if the patients are in remission at the time of HSCT. The long-term disease free survival rate for patients with relapsed AML and no HSCT is 5-10%. Many relapsed patients have refractory chemoresistant disease and never attain remission to be eligible for potentially curative HSCT, or develop significant complicating comorbidities during the prolonged intensive reinduction of their disease. Thus, improved strategies for achieving remission in relapsed patients prior to transplantation are critical to improving the survival of these patients.

### 1.2 Reinduction Chemotherapy for AML

Reinduction chemotherapy in relapsed AML results in highly variable remission rates partly because of heterogeneity in this population. A metareview of 31 trials over the past 20 years revealed no single superior regimen. In one study, the mean second complete remission (CR2) rate for high-risk patients (those for which the first complete remission (CR1) was less than 1y) was 27.6%+/−15.5 (weighted mean+/−SD), whereas for low-risk patients (patients with CR1 equal or greater than 1y) the CR2 rate was 56.1%+/−25.9. In another study, patients with high-risk disease (primary refractory disease or CR1 less than 6 months) had a CR2 rate of only 10%, compared to over 50% for those with low-risk disease (CR1 equal or greater than 18 months), and patients with good-prognosis karyotype achieve a second or third remission more often than those with poor-prognosis karyotype.

The importance of high-dose cytosine arabinoside (cytarabine, Ara-C) as an integral agent in primary and salvage regimens for the treatment of AML has been well-established. Fludarabine has been widely used to lymphodeplete patients prior to infusion of lymphocytes, and fludarabine-containing regimens, usually combined with cytarabine with or without an anthracycline, have been used for reinduction of primary refractory or relapsed AML. It was demonstrated that fludarabine potentiates in AML blasts an increase in intracellular retention of Ara-CTP, the active metabolite of cytarabine. This led to development of the highly active FLAG (fludarabine, cytarabine, G-CSF) regimen for AML.

FLAG chemotherapy as originally described has exhibited excessive toxicity in patients over age 60, but has been safely delivered in clinical trials to this age group when fludarabine and cytarabine are reduced from 5 days to 4 days.

### 1.3 the Use of Colony Stimulating Factors in the Treatment of AML

Colony stimulating factors for granulocytes (G-CSF) and granulocytes-macrophages (GM-CSF) enhance neutrophil recovery following high-dose chemotherapy. The use of G-CSF during induction therapy for AML results in superior event-free survival. In addition, they increase the sensitivity of myeloid leukemic stem cells to cytarabine by augmenting accumulation of Ara-CTP, and have therefore been used to augment the anti-leukemic effect of combination chemotherapies such as FLAG. Moreover, GM-CSF has been shown to enhance the activity of NK cells against AML blasts in vitro and in the setting of autologous transplant.

### 1.4 Human NK Cells as Mediators of Anti-Tumor Therapy

Human NK cells are a subset of peripheral blood lymphocytes typically defined by the expression of CD56 or CD16 and the absence of the T-cell receptor CD3. A number of studies suggest that NK cells have a role in tumor surveillance. Cell lines susceptible to NK lysis are designated “NK sensitive” targets. The prototype NK sensitive target is the leukemia cell line K562. Activation of NK cells with cytokines, in particular IL-2, gives NK cells the ability to lyse tumor targets not normally sensitive to NK lysis (NK resistant targets).

NK cells are regulated by KIR receptor-ligand interactions and are cytotoxic against certain HLA class I mismatched targets. Alloreactive HLA haploidentical NK cells in the SCT setting have been reported to enhance engraftment, reduce GvHD and prevent relapse of leukemia. Infusion of human haploidentical NK cells without hematopoietic transplantation in patients with AML have been studied. The cells were given after cytoreductive chemotherapy to induce lymphocytopenia and support homeostatic expansion of the NK cells after infusion. The NK cells were obtained by leukapheresis of the donor with subsequent depletion of CD3+ T-cells, with or without secondary positive selection of CD56+ cells, which were then activated overnight with IL-2.

Infusions of up to 2×10⁷ NK cells/kg were well tolerated and produced remission in 5 of 15 patients with refractory AML. Graft-vs-host disease and protracted pancytopenia did not occur. Donor cells were detectable for up to 4 weeks.

The poor anti-tumor effect by autologous NK cells in previous trials may be due to several factors including the resistant nature of tumors, factors released by the tumor, and killer immunoglobulin receptors (KIR). The effectors mediating graft-versus-host disease (GvHD) and graft-versus-tumor (GVT) are still uncertain, but some murine models suggest that GVT activity in vivo correlates strongly with NK activity in vitro. Using an allogeneic transplant model against the A20 leukemia cell line, allogeneic NK infusions were protective against leukemia relapse and had no adverse effect on leukocyte engraftment. In vitro cytotoxicity assays have shown superior lytic potential of allogeneic IL2-activated NK cells compared to syngeneic or autologous NK cells. Transplantation of syngeneic or autologous lymphocyte-depleted infusates without NK cell infusions resulted in 10 and 15% disease-free survival rates. However, adoptive transfer of IL2-activated syngeneic NK cells improved survival to 50%. In contrast, treatment with allogeneic NK cells resulted in an even stronger anti-tumor effect with 85% of animals surviving disease-free. The hypothesis that Class I induced inhibition of NK cell lysis is important in anti-tumor therapy is strongly supported by these in vivo murine experiments showing allogeneic NK cells exhibit greater in vivo anti-tumor activity than autologous NK cells.

### 1.5 Selecting KIR Mismatched Donors and Recipients

NK cells recognize “self” on autologous targets through HLA class I associated KIR. This process suppresses NK cell lysis of targets. There are four inhibitory KIR genes found to be relevant for NK alloreactivity with known HLA specificity: 2DL1 binds to HLA-C group 2 alleles, 2DL2 and 2DL3 bind to HLA-C group 1 alleles, and 3DL1 binds to HLA-B Bw4 alleles. According to the missing-ligand model for each KIR gene present there will be alloreactivity only if the corresponding ligand is absent in the patient, and is present in the donor. Example data for Caucasian donors is illustrated in Table 3, which summarizes the analysis of HLA Bw and C group loci and KIR expression for donor GVL alloreactivity, below. C1/C2/Bw4 alleles occur in 32% of the population. Of the 23 KIR genotypes that account for 80% of the population, 25.3% meet all of these criteria. ˜90% of adults will have been exposed to CMV. Thus, the “ideal” NK cell donor can be identified in approximately 1 out of 16 healthy individuals.

TABLE 3
Summary of HLA Bw and C group loci and KIR expression
analysis for donor GVL alloreactivity.

| | Donor | | | | | | |
|---|---|---|---|---|---|---|---|
| | | C1*C1 | C1*C2 | C2*C2 | C1*C1*Bw4 | C1*C2*Bw4 | C2*C2*Bw4 |
| Recipient | C1*C1 | No GvL | GvL | GvL | GvL | GvL | GvL |
| | C1*C2 | No GvL | No GvL | No GvL | GvL | GvL | GvL |
| | C2*C2 | GvL | GvL | No GvL | GvL | GvL | GvL |
| | C1*C1*Bw4 | No GvL | GvL | GvL | No GvL | GvL | GvL |
| | C1*C2*Bw4 | No GvL | No GvL | No GvL | No GvL | No GvL | No GvL |
| | C2*C2*Bw4 | GvL | GvL | No GvL | GvL | GvL | No GvL |

Alloreactivity in the GVL direction is likely to occur in the indicated combinations. This study selects the donor with the greatest likely KIR reactivity in the GVL direction using this model. Donors having C1, C2, and Bw4 HLA are maximally mismatched to provide GvL to the greatest number of recipients.

i.

### 1.7 Ex Vivo Expansion of NK Cells

The major obstacle for adoptive NK cell immunotherapy is obtaining sufficient cell numbers, as these cells represent a small fraction of peripheral white blood cells, propagate poorly ex vivo, and have limited life spans in vivo. Common gamma-chain cytokines are important in NK cell activation, maturation, and proliferation. Others have described improved ex vivo expansion with soluble cytokines, artificial antigen presenting cells (aAPC), and aAPC engineered with costimulatory molecules and/or membrane-bound IL-15 (mIL-15). Our group generated a membrane-bound IL-21 fusion protein (mIL21), and found superior ex vivo expansion of NK cells when stimulated with K562 aAPC genetically modified to express mIL21 and the costimulatory molecules CD86 and CD137L. Freshly isolated peripheral blood mononuclear cells (PBMC) are co-cultured with irradiated K562 aAPC at a ratio of 2:1 (aAPC:PBMC) in the presence of 50 IU/ml of rhIL-2 and then re-stimulated with aAPC every seven days at ratio of 1:1.

K562-mIL21 aAPCs were able to promote a mean NK-cell expansion of 37,200-fold by day 21, with 85% of donors achieving at least 5,000-fold expansion (see also Example 1). Expanded cells expressed very high CD16 levels, NCR levels, and retained the pre-expansion KIR repertoire. These cells showed high cytotoxicity to tumor targets and ADCC participation.

Thus, clinically significant NK cell expansion from small peripheral blood samples is possible using aAPCs expressing mIL21.

### 1.8 Purpose of Clinical Trial

Relapsed AML requires remission prior to allogeneic HSCT for optimal survival, but is a disease with poor response to chemotherapy. HLA-haploidentical, NK-enriched peripheral blood cell infusions have shown safety in patients with poor prognosis AML. Though not powered for such an assessment, this trial showed a promising but not statistically significant trend in remission rate. NK cell therapy for AML, especially relapsed AML is limited by small numbers of NK cells attainable through leukapheresis. AS described herein, large numbers of NK cells can however be propagated ex vivo from a small volume blood draw, alleviating the need for donor leukapheresis.

The purpose of this trial is to determine the safety, feasibility and maximum tolerated dose of mIL21-expanded haploidentical NK cells in conjunction with FLAG chemotherapy in patients with relapsed/refractory AML.

### 2.0 Eligibility

### 2.1 Patient Inclusion Criteria

1. Patients with relapsed or primary refractory AML. Patients with relapsed AML after allogeneic stem cell stransplantation, including those who have received donor lymphocyte infusions, are eligible if they have no active GvHD and are off immunosuppression.

2. Have a haploidentical family peripheral blood donor selected for best possible KIR reactivity.

3. Patient age>/=18 years old.

4. Performance status: Karnofsky or Lansky Performance Scale (PS) greater or equal to 70.

5. Renal function: Serum creatinine <2 mg/dl or creatinine clearance greater or equal than 40 cc/min. Creatinine for pediatric patients <2 mg/dl or <2 times upper limit of normal for age (whichever is less).

6. Pulmonary function: FEV1, FVC and DLCO >50% of expected, corrected for hemoglobin. For pediatric patients, if unable to perform pulmonary function tests (most children <7 years of age), pulse oximetry>/=92% on room air by pulse oximetry.

7. Liver function: Total bilirubin <2 mg/dl or <2.5×ULN for age (unless Gilbert's syndrome) and SGPT (ALT)<2.5×ULN for age.

8. Cardiac function: left ventricular ejection fraction >40%. No uncontrolled arrhythmias or uncontrolled symptomatic cardiac disease.

9. Negative serum test to rule out pregnancy within 2 weeks prior to registration in females of childbearing potential (non childbearing potential defined as premenarchal, greater than one year post-menopausal, or surgically sterilized).

10. Sexually active males and females of childbearing potential must agree to use a form of contraception considered effective and medically acceptable by the Investigator.

11. Negative serology for human immunodeficiency virus (HIV).

### 2.2 Patient Exclusion Criteria

1. Investigational therapies in the 4 weeks prior to beginning treatment on this protocol.

2. Congestive heart failure <6 months prior to screening.

3. Unstable angina pectoris <6 months prior to screening.

4. Myocardial infarction <6 months prior to screening.

5. Uncontrolled infection, defined as an infection which has not resolved spontaneously or does not show evidence of significant resolution after initiating appropriate therapy, excluding chronic asymptomatic viral infections (e.g., HPV, BK virus, HCV, etc.).

### 2.3 Donor Eligibility Criteria and Evaluation Prior to Donation

1. Donor must be 16 years of age or older and weigh at least 110 pounds.

2. Donor must be an HLA-haploidentical relative selected for best NK alloreactivity, defined as having a KIR gene present on the Donor NK cells for which the relevant HLA haplotype (KIR ligand) is absent in the Recipient and present in the Donor or selected on the basis of activating KIR gene content.

3. Donor must meet standard institutional eligibility and donor certification criteria for therapeutic cell product donation.

4. Not be pregnant as defined by negative serum (beta HCG) pregnancy test in females of childbearing potential (Non-childbearing potential defined as premenarchal, previous surgical sterilization, or postmenopausal for >12 months).

5. Evaluation:
History and physical examination.
Laboratory examinations: hematology, electrolytes, chemistry.
Infectious disease screening and serology.
HLA and KIR typing.

### 3.0 Treatment Plan

In this study the first NK cell infusion is referred as day zero (D0), treatment plan activities prior or after D0 are denominated as day minus (D−) or day plus (D+).

### 3.1 Donor Peripheral Blood NK

One unit (approximately 500 mL) of peripheral blood will be drawn from the donor to start the NK cell expansion on aAPC for 14 days.

### 3.2 FLAG Treatment Administration Following Standard of Care Practice.

After collection of donor peripheral blood for NK cell expansion, recipient may begin FLAG chemotherapy as soon as deemed appropriate by the treating physician. G-CSF will be given daily beginning one day prior to first dose of fludarabine/cytarabine and continuing until post nadir absolute neutrophil counts (ANC) are equal or over 1000. G-CSF may be held for high peripheral blast counts at physician discretion for patient safety. Fludarabine will be administrated at 30 mg/m²/day for five days, the dose based on actual BSA calculated from actual body weight and height. Approximately four hours later Cytarabine will be administrated at 2 g/m²/day for five days. Patients over age 60 will receive dose modification by receiving only 4 days of fludarabine and cytarabine.

Rest period for 2-14 days before NK cell infusion.

### 3.3 NK Cell Infusion on Days 0 to 14 for 6 Doses Total According to Dose Escalation Schema.

NK cell infusions may begin as soon as release criteria are met for the expanded cells, to start no less than 2 days and no more than 15 days after the last dose of fludarabine/cytarabine. NK cells will be delivered 3 times a week, over at least a four-day period (e.g., MWF, MTuTh, TuThF, etc.). NK cells will be infused according to SCTCT Department SOP for therapeutic cell infusions.

Anaphylactic Medications: Prior to NK cell infusion, have the following medications IMMEDIATELY available. Give and call MD if anaphylaxis occurs.

Epinephrine (1:1000) 0.5 mL to be administered subcutaneously

Diphenhydramine 50 mg to be administered intravenously

Must discuss with MD prior to administration of corticosteroids in case of anaphylaxis.

Follow MDACC HSR algorithm for additional supportive care measures.

Premedications: Prior to infusion of NK cells. Diphenhydramine 25 mg to be administered intravenously.

The first NK cell dosing cohort will be well below the currently-established safe dose of apheresis-derived NK cells, as expanded NK cells may have increased toxicity because of their activated phenotype. In order to avoid accruing patients at suboptimal doses, a dose escalation schema will be followed.

| | NK cell dose/ | Total NK | Maximum total |
|---|---|---|---|
| Cohort | infusion | cell dose | T cell dose |
| 1 | 10⁶/kg | 6 × 10⁶/kg | 10⁵/kg |
| 2 | 3 × 10⁶/kg | 1.8 × 10⁷/kg | 10⁵/kg |
| 3 | 10⁷/kg | 6 × 10⁷/kg | 10⁵/kg |
| 4 | 3 × 10⁷/kg | 1.8 × 10⁸/kg | 10⁵/kg |
| 5 | 10⁸/kg | 6 × 10⁸/kg | 10⁵/kg |
| 6 | 3 × 10⁸/kg | 1.8 × 10⁹/kg | 10⁵/kg |

The principles of an rapid dose escalation method are used in this study to allow expeditious advancement up to the current safe dose of NK cells.

To be able to receive the NK infusion(s) patients must meet the following requirements:

1. Off corticosteroids within prior 72 hour period.

2. Not requiring ventilator support or supplemental oxygen.

3. Performance status Karnofsky or Lansky greater or equal to 70%.

The NK cell dose will be based on total nucleated cell (TNC) count and flow cytometry assessment of CD56+CD3− percentage. The maximum volume of cell product infused is 100 ml. The cells infused will be delivered on the basis of NK cells/kg recipient weight Total CD3+ T cells must be less than 1×10⁵/kg recipient weight for all cohorts. If infusing the number of NK cells for the current cohort will result in delivering >10⁵ CD3+ cells/kg recipient weight, the NK cell dose for infusion will be reduced to that of the highest cohort at which the infused CD3+ cells will be <1×10⁵/kg recipient weight. Some donor NK cell expansions may not yield sufficient cells to reach the planned NK cell dose. If the target NK cell/kg recipient weight cannot be delivered, then the NK cell dose for infusion will be reduced to the highest cohort achievable. The patient data will be included on that cohort for statistical analysis, and the current dose level will enroll an additional subject.

### 4.0 Drug Information

4.1 Cytosine arabinoside (CYTarabine, Ara-C)

Cytarabine is an antimetabolite. Cytarabine for injection is commercially available as a solution. Institutional guidelines for handling, reconstitution and administration should be followed. Cytarabine can cause cardiomegaly, coma, neurotoxicity (dose-related, cerebellar toxicity may occur in patients receiving high-dose cytarabine [>36-48 g/m²/cycle]; incidence may up to 55% in patients with renal impairment), personality change, somnolence, alopecia (complete), desquamation, rash (severe), gastrointestinal ulcer, peritonitis, pneumatosis cystoides intestinalis, hyperbilirubinemia, liver abscess, liver damage, necrotizing colitis, peripheral neuropathy (motor and sensory), corneal toxicity, hemorrhagic conjunctivitis, pulmonary edema, syndrome of sudden respiratory distress, and sepsis.

Formulation: 100, 500, 1000, or 2000 mg vial as a solution for IV use.

Commercially available.

Storage: Room temperature.

Stability: 28 days at room temperature.

Administration: Cytarabine is further diluted in 5% dextrose or 0.9% sodium chloride.

### 4.2 Fludarabine

Fludarabine is an antimetabolite. Fludarabine for injection is commercially available as a lyophilized cake that is reconstituted in sterile water. Institutional guidelines for handling, reconstitution and administration should be followed. Fludarabine can cause lowering of blood counts, suppression of the immune system, nausea and vomiting, fever, hypersensitivity reaction, tumor lysis, transient elevation in serum transaminases, hemolysis, and neurotoxicity at doses higher than administered in this study

Formulation: 50 mg vial as a white lyophilized cake for IV use. Commercially available.

Storage: Room temperature.

Mixing: Add 2 mL sterile water to vial to give a final concentration of 25 mg/mL.

Stability: I.V. solution should be used within 8 hours of mixing.

Administration: Fludarabine is further diluted in 100 mL of 5% dextrose or 0.9% sodium chloride.

### 4.3 Filgrastim (G-CSF; Granulocyte Colony Stimulating Factor)

Filgrastim stimulates the production, maturation, and activation of neutrophils. It also activates neutrophils to increase both their migration and cytotoxicity. It is used in chemotherapy-induced neutropenia (nonmyeloid malignancies, acute myeloid leukemia, and bone marrow transplantation); severe chronic neutropenia (SCN); patients undergoing peripheral blood progenitor cell (PBPC) collection.

It has been associated with:
Allergic reactions: Rash, urticaria, wheezing, dyspnea, tachycardia, and/or hypotension have occurred with first or later doses. Reactions tended to occur more frequently with intravenous administration and within 30 minutes of administration.
Respiratory distress syndrome: Rare cases of adult respiratory distress syndrome have been reported; patients must be instructed to report respiratory distress.
Spleen rupture: Rare cases of spleen rupture have been reported; patients must be instructed to report left upper quadrant pain or shoulder tip pain.

### Pharmacodynamics/Kinetics

Onset of action: 24 hours; plateaus in 3-5 days

Duration: ANC decreases by 50% within 2 days after discontinuing G-CSF; white counts return to the normal range in 4-7 days; peak plasma levels can be maintained for up to 12 hours

Absorption: SubQ: 100%

Distribution: 150 mL/kg; no evidence of drug accumulation over a 11- to 20-day period

Metabolism: Systemically degraded

Half-life elimination: 1.8-3.5 hours.Time to peak, serum: SubQ: 2-6 hours

Dosage: SubQ: <5 mcg/kg/day beginning 24-72 hours following chemotherapy; continue until absolute neutrophil count reaches target. Pediatric patients should receive specific calculated dose. Adult doses should be rounded off to the nearest vial size (300 mcg or 480 mcg)

Autologous stem cell collection: 5 mcg/kg SubQ every 12 hours for 5 days (10 doses total)

Dosage Formulation:

Injection, solution: 300 mcg/mL (1 mL, 1.6 mL)

Injection, solution [prefilled syringe]: 300 mcg/0.5 mL

### 5.0 Study Evaluations

5.1 Prior to starting FLAG standard treatment (Baseline):
5.1.1 History and Physical Examination.
5.1.2 CBC with differential.
   5.2 Before each NK infusion:
6.2.1 History & Physical Examination.
6.2.2 CBC with differential.
6.2.3 Pulse oximetry.
   5.3 After last NK infusion: CBC with differential twice a week while patients are neutropenic
   5.4 After neutrophil recovery: CBC with differential once a week until D+56 from NK infusion #1.
   5.5 Disease assessment: after neutrophil recovery and or around D+28, whichever is earlier:
1. Unilateral bone marrow biopsy and aspirate for cytology, flow cytometry, MRD, chimerism (STR or FISH), cytogenetics, and FISH (for known tumor markers).
2. If recovery has not occurred by Day +28, then a second bone marrow will be obtained at the time of neutrophil recovery or around Day +56, whichever is earlier.
   5.6 Peripheral blood to address study secondary objectives to be sent to Dr. Lee's Laboratory (MOD1.020).
1. Prior to start FLAG standard treatment (Baseline).
2. Before and 2 hrs (+/−1 hr) after completion of each NK infusion.
3. D+14 (+/−3 days), +16 (+/−3 days), +18 (+/−3 days), and +21 (+/−3 days), then weekly until D+56 while infused NK cells can be reliably detected. Samples can be obtained plus/minus 3 days before D+28 and plus/minus 5 days after D+28 of the target date. For each sample, draw up to 40 mL (0.5 mL/kg max) in Na-Heparin green-top tube and up to 10 mL of serum (1 red top tube).

### 6.0 Adverse Events

### 6.1 Assessment of the Adverse Events Attribution

The investigational component of the treatment plan of this study is the NK cell infusion. FLAG chemotherapy and GCSF are considered standard of care and their associated adverse events are well known. Therefore, for the purpose of this study when, in the presence of an adverse event which a direct relationship to the NK cell infusion is suspected, the event will be attributed to the NK cell infusion.

Events known to be caused by FLAG chemotherapy and their direct consequences, as well as those events known to be related to drugs used for the treatment of GvHD, infections, and supportive treatment will be scored as unrelated to the NK cell infusion.

The principal investigator will be the final arbiter in determining the attribution of the event.

### 6.2 Assessment of the Adverse Events Severity.

The severity of the adverse events (AEs) will be graded according to the Common Terminology Criteria v4.0 (CTCAE) from the start of the first NK cell infusion up to D+56.

Events not included in the CTCAE chart will be scored as follows:

General grading:
Grade 1:
Mild: discomfort present with no disruption of daily activity, no treatment required beyond prophylaxis.
Grade 2:
Moderate: discomfort present with some disruption of daily activity, require treatment.
Grade 3:
Severe: discomfort that interrupts normal daily activity, not responding to first line treatment.
Grade 4:
Life Threatening: discomfort that represents immediate risk of death.
   6.3 Expected Adverse Events possibly associated with infusion of allogeneic NK cells:

1. Acute Adverse Events:

Events lasting less than 24 hours:

Grade I chills

Grade I cough

Grade I or II angioedema

Grade I or II dyspnea

Grade I or II hypotension

Grade I or II tachycardia

Grade I or II headache

Events lasting less than 48 hours:

Grade I or II fatigue

Grade I or II neuropathic pain

Grade I or II vomiting

Grade I or II SGPT changes

Grade I or II hypoalbuminemia

Grade I or II hypocalcemia

Grade I or II fever

Grade I or II pruritis

Grade I rash

Grade I or II lymphopenia

Grade I or II neutropenia

Grade I or II leukopenia

Grade I or II cytokine release/acute infusion reaction

2. Events Lasting Less than 72 Hours:

Grade I or II nausea

Tumor Lysis Syndrome

3. Cytopenias after 2 to 3 Weeks Post First NK Cell Infusion.

Fludarabine and cytarabine are expected to cause transient marrow suppression lasting 2-3 weeks. However, hematologic toxicity due to allogeneic NK cells may occur later, and therefore hematologic recovery will be assessed beyond the expected chemotherapy-induced nadir. For example, 10 to 15% of patients receiving donor lymphocyte infusion after allogeneic HSCT develop marrow suppression.

Cytopenia in this setting is usually attributed to T-cell suppression of host hematopoietic cells. Although this situation is unlikely after infusion of T-cell depleted NK-cell infusions, the possibility of NK-mediated marrow suppression cannot be ruled out prospectively. In addition, the time for recovery of normal hematopoiesis is highly dependent on the presence of normal marrow reserves, which may be nearly absent in the setting of multiply-relapsed and heavily treated patients.

4. Acute Graft-Versus-Host Disease.

GvHD is associated with allogeneic T cells. Since the infused cells will be subjected to T-cell depletion, GvHD is not expected, and has generally not occurred in previous trials using allogeneic NK cell therapy. However, small numbers of T cells may be infused or NK cells may engraft and cause GvHD syndrome.

It is unexpected that GvH above overall grade 2 to occur.

Adverse events considered serious.

1. Treatment refractory GvHD.

2. Infections during the neutropenia period requiring hospitalization.

3. Any expected or unexpected event considered related to the NK cell product resulting in an irreversible condition and/or leading to death.

Expected Adverse Events Known to be Associated with FLAG Chemotherapy.

Toxicities known to occur with the combination of fludarabine, cytarabine, and G-CSF (FLAG) are well described from prior published phase 1 and 2 trials. Expected toxicities that are first noted after initiation of FLAG and before administration of NK cells, and bone marrow suppression, cytopenias, and infections will not be attributed to the NK cells for the purpose of determining DLT.

Adverse Events (% Grade III and IV) Associated with FLAG:

1. Liver:

ALT (25%), Bilirubin (7%), AST (7%), Alkaline phosphatase (5%).

2. GI Track:

ALT (25%), Mucositis (5%), Nausea/vomiting (30%), Diarrhea (6%), Constipation (4%).

3. Other:

Hemorrhage (5%), Rash (5%), BUN (4%), Drug fever (3%), Headache (3%) and vision changes (1%).

4. Bone marrow suppression and associated cytopenias with a median time to recovery (95% CI) from Day 0 of chemotherapy: Neutrophil 32 (27-35) days, platelet 41 (35-47) days.

5. While neutropenic period, patients are at risk for infections.

### Adverse Events Data Collection.

From D0 up to D+56 the collection of adverse events will reflect the onset and resolution date and maximum grade. Intermittent events should be labeled as such and followed until resolution.

If a patient is taken off study while an event is still ongoing, this will be followed until resolution unless another therapy is initiated. Pre-existing medical conditions will be recorded only if an exacerbation occurs during the active treatment period. Co-morbid events will not be scored separately.

Adverse events will be documented based on progress notes, including the flowsheet, in the electronic (Clinic Station) patient medical record.

PDMS/CORe will be used as the electronic case report form for this protocol and all protocol specific data will be entered into PDMS/CORe.

### Concurrent Medication.

Patients treated on this protocol will require supportive care treatment (concurrent medications). These medications are considered standard of care and have no scientific contributions to the protocol, therefore no data will be captured on the various medications needed or their side effects.

### 7.0 Statistical Considerations

The primary objective of this study is to evaluate the safety and feasibility and define the maximum tolerated dose (MTD) of an expanded haploidentical donor NK cell product following a FLAG preparative regimen to treat relapsed/refractory acute myelogenous leukemia. The endpoint for maximum tolerated dose of NK cell infusion is described herein. The endpoint of safety and feasibility is defined as being able to generate and infuse NK cells at the maximum tolerated cell dose without exceeding toxicity limits, in greater than or equal to 7 of 10 subjects. The secondary endpoints include assessing the activation status and the persistence of haploidentical NK cells, the immunophenotype and function of haploidentical NK cells, the rate of remission of AML disease, the rate at which patients receiving this regimen are able to undergo transplant, and the time-to-transplantation for those with available donors.

The cytokine-mediated activation of NK cells will be determined by flow-based activation assay determining CD107a expression of NK cells in response to standardized targets. The function of NK cells will be assessed by cell lysis of standardized targets. Remission will be defined as marrow recovery with <5% blasts in the bone marrow. Clinical responses will be correlated with NK cell expansion in vivo, cytokine levels, expression of activation markers, and expression of NK cell ligands on the patients AML blasts. Additional research samples will be collected at the indicated time points for laboratory evaluation of in vivo activation of the expanded NK cells to study the effect of this therapy on the immune system. Toxicity and the occurrence of adverse events will be monitored.

### 7.1 Dose Escalation

A dose-limiting toxicity (DLT) is defined as:
1. >Grade 3 infusional allergic reaction related to the NK cells infusion.
2. >Grade 3 acute overall GvHD that does not resolve with treatment to <=Grade 1 within one week.
3. >Grade 3 unexpected toxicity possibly, probably, or definitely related to the NK cell infusion. Grade 3 toxicities that resolve within 72 hours will not be counted as a DLT.

Since NK cells delivered at doses equivalent to dose levels 1-4 have been shown to be safe in other phase I trials, we will utilize a rapid dose escalation method through those dose levels. We will use the standard 3+3 design for dose levels 5-6. Once the 3+3 portion of the study is implemented, concurrent enrollment at any dose level will be limited to the minimum number of subjects needed to declare the MTD exceeded (e.g., a dose level may begin with two subjects enrolled concurrently, but to enroll a third subject, at least one of the first two subjects must be observed through Day +28 without a DLT.

For dose levels 1-4, one patient will be treated at each dose level 1 (10\hat{ }6/kg/dose, thrice weekly×6 doses). If this patient does not exceed the toxicity limits defined for the rapid escalation phase (see first bullet point below), then the next patient will be treated at the next dose level. If at any time in dose levels 1-4 a Grade 2 or greater related toxicity as described is observed, the standard 3+3 will immediately start and an additional 2 patients will be enrolled at the current dose level. If the 3+3 has not started through the first 4 doses, the standard 3+3 design will start for dose level 5 (10\hat{ }8/kg/dose). Three patients will be treated and evaluated for toxicity. If 0/3 patients experience DLT, the next cohort of 3 patients will be treated at the next higher dose level. If 1 of 3 patients treated at a dose level experiences DLT, then 3 more patients will be treated at the same dose level. If the incidence of DLT among those 6 patients is 1 in 6, then the next cohort is treated at the next higher dose level. If more than 2 of 6 patients treated at a dose level experience DLT, then the MTD is considered to have been exceeded. Three more patients will be treated at the next lower dose as described above unless 6 patients have already been treated at that dose. The MTD is defined as the highest dose studied in which 6 patients have been treated and at most 2 patient with DLTs is observed. If 2 of 6 DLTs are observed, stop and declare that dose level as the MTD.

The cohort defined as the MTD may be expanded to up to 10 patients to further evaluate toxicity and correlative data. During the expansion, if at any time >⅓ of patients experience a DLT, the expansion cohort will be terminated. If the MTD expansion cohort is terminated due to excessive toxicity, the next lower dose may be expanded to 10 and explored. All patients treated at the MTD will be included in the expansion analysis and monitoring.
During the rapid escalation phase, a more stringent criteria for toxicity will be utilized to ensure patient safety. Occurrence of an NK cell product-related grade 2 toxicity, excluding grade 2 fever, rigor/chills, fatigue, vomiting/nausea, pruritus/itching, electrolyte imbalance, hypoalbuminemia and lymphopenia, by any one patient within 21 days from the start of NK cell product infusion: Expand the current and subsequent (if any) cohorts to include up to 3 patients.
If the MTD is not established by dose level 6, this dose level will be expanded to 10 patients to further assess the safety of and antitumor response to treatment with expanded NK cells.
If at any time during the cohort expansion the stopping rules apply, the patient enrollment in this expansion cohort will be suspended.
After the last patient in a cohort has completed the treatment, clinical and safety data will be analyzed and the dose escalation will be governed by the dose escalation rules defined above.
MTD—Maximum Tolerated Dose is defined as the highest dose level at which no more than two patients in a 6-patient cohort experience a DLT during treatment. If 2 of 6 DLTs are observed, stop and declare that dose level as the MTD.

### 7.2 Trial Size Justification

Up to 6 patients per cohort may be enrolled during the dose escalation phase of the trial. Following determination of the maximum tolerated dose of NK cells, we will enroll subjects until we have 10 subjects on study with successful NK-cell infusion at the MTD level or the highest dose level. We expect to accrue these patients over 2 years. Patients who fail to meet criteria to receive the NK cell infusion will not be included in determining the primary objective of feasibility. For each enrolled patient that did not receive an NK-cell infusion at the scheduled dose level, an additional patient will be enrolled. We anticipate up to 6 patients may not be able to receive the NK cells at the MTD or the highest dose level because of toxicity of the FLAG regimen. Thus, the trial may complete dose level 6 with as few as 17 subjects, or may enroll up to 46 subjects.

A secondary aim of this study will be the assessment of complete remission (CR) at day 56 following infusion of the NK cells. For efficacy, we will assess outcome based on patient risk. The historical remission rate for relapsed AML across multiple regimens is 56.1% for low-risk patients, and 27.6% for high-risk patients.

### 7.3 Study Stopping Rules

Adverse events will be defined according to NCI CTC AE v4.0 criteria.
If more than 2 subjects experience >Grade 4 adverse events that are possibly, probably, or definitely attributed to the infused NK-cell product involving cardiopulmonary, hepatic (excluding albumin), neurologic, or renal systems, or severe (>Grade 4) infections, we will temporarily close new patient entry to this trial to review the possible need for modifications to the safety criteria and/or consent forms.
If any death possibly, probably or definitely attributed to the infused NK cells occurs in a research participant within 30 days of the NK cell infusion, we will temporarily close new patient entry to this trial to review the possible need for modifications to the safety criteria and/or consent forms. Deaths occurring more than 30 days after the NK cell infusion will only result in temporary termination and review of the study if the death is definitely attributable to the NK cell therapy.

### 7.4 Analysis of Secondary Study Endpoints

7.4.1 Analysis of NK-cell numerical expansion in vivo:

Peripheral blood will be obtained before therapy, during the NK cell treatment period, and after NK cell treatment. The studies may include flow cytometry analyses and sorting and molecular studies. Donor NK-cell expansion will be defined as an absolute circulating donor-derived NK cell count that increases above the post-infusion level. The following chimerism methods will be employed to determine origin and number of circulating NK cells:

7.4.2 Chimerism studies:
Chimerism may be determined by flow cytometry using haplotype-specific antibodies.
Chimerism may be determined by STR polymorphisms.
When there is a sex-mismatch between the donor and the recipient, assays based on determining the frequency of sex-chromosomes may be used. Testing may be altered by Principal Investigator or designee.

### 7.5 Clinical Outcomes

We will use descriptive statistics to summarize the demographic and clinical characteristics of the patients on this study. We will estimate the complete remission rate (CR) and time to transplantation (TTT) with the Kaplan-Meier estimator and tabulate with 95% confidence intervals. We will estimate the CR and TTP with a 95% confidence interval. We will estimate the proportion of patients with successful in vivo NK-cell expansion with a 95% confidence interval. We will use Cox proportional hazards regression to model CR and TTT as a function of NK cell dose.

### 7.6 Accrual Estimates

We expect a minimum of 15 eligible patients per year to be enrolled. This protocol may take up to 3 years to complete.

### 8.0 Study Criteria

8.1 Recovery: defined as the first day of sustained ANC equal or over 1000/uL.
8.2 Prolonged Neutropenia: failure to reach recovery within 28 days after the infusion of the NK cells.
8.3 Progression of disease: detection of persistent or progressive underlying disease by bone marrow and or peripheral blood examination.

### 8.4 Off Study:

8.4.1 Inability to infuse the NK cell product due to product contamination or insufficient cell dose.

8.4.2 Graft failure requiring further treatment.

8.4.3 Disease progression requiring further treatment.

8.4.4 Patient responds to treatment and goes on to receive other therapy (e.g., stem cell transplant).

8.4.5 Unexpected pattern of toxicity.

8.4.6 Patient withdrawal of the informed consent.

8.4.7 Patient is noncompliant with treatment schema.

8.4.8 After treatment completion, D+56.

### Example 3: Cytotoxicity of Natural Killer Cells Expanded from PBMC's from a Universal Donor

NK cells are prepared by an expanding from PBMC's obtained from a universal donor identified by the method described in FIG. 3. Expansion is performed in the presence of membrane-bound IL-21 in the form of irradiated feeder cells with membrane bound IL-21, plasma-membrane particles bearing IL-21, or exosomes bearing IL-21. PBMCs are first isolated from buffy coat, grown in a cell medium supplemented with 10% FBS and maintained at 37° C. in a humidified atmosphere with 5% C02. Starting on day 5 of culture, media is exchanged every other day by replacing half of the media with fresh media supplemented with 100 U of IL-2. Cells are counted every other day and the culture content checked regularly starting on day 7. NK cells are expanded over a period of at least 7-14 days. Cytotoxicity assays are performed as follows: ovarian cancer derived target cell line SKOV3 transfected for green fluorescent protein (GFP) is used as a target to measure anti-tumor cytotoxicity of effector NK cells expanded from universal donor PBMC's. Target cells are cultured alone (control wells) or co-cultured with NK Cells for 45 minutes in a 37° C., 5% C02 atmosphere. The cells are then centrifuged and resuspended in a labelling buffer containing antibody, and incubated for prior to analysis by flow cytometry. The cytotoxicity is determined based on the absolute amount of Viable Target Cells (GFP+/Antibody−) remaining in each well and referenced to average VTC in “target alone” control wells.

CytotoxicityE:T(%)=(VTCE:T/Average VTCT ctrl.)*100

The cytotoxicity of NK cells expanded from PBMC's obtained from a universal donor are found to have increased cytotoxicity toward SKOV3 cells, relative to NK cells expanded from PBMC's obtained from a control donor that does not satisfy the universal donor criteria provided herein.

### Example 4: Treatment Using NK Cells Expanded from PBMC's from a Universal Donor

At least 15 AML patients are selected as described in example 2, and treated according to the clinical trial protocol detailed in Example 2 (Section 3), over a period of about 3 years, using NK Cells derived from a universal donor, and expanded according to Example 3. Peripheral blood from each patient is obtained before therapy, during the NK cell treatment period, and after NK cell treatment. Flow cytometry analyses and sorting and molecular studies are performed during treatment. Complete remission rate (CR) and time to transplantation (TTT) are determined with the Kaplan-Meier estimator and tabulated with 95% confidence intervals. CR and TTP are determined with a 95% confidence interval. The proportion of patients with successful in vivo NK-cell expansion is determined with a 95% confidence interval. Cox proportional hazards regression is used to model CR and TTT as a function of NK cell dose. Recovery is defined as the first day of sustained ANC equal or over 1000/uL. Prolonged Neutropenia is defined as failure to reach recovery within 28 days after the infusion of the NK cells. Progression of disease is determined upon detection of persistent or progressive underlying disease by bone marrow and or peripheral blood examination. A majority of AML patients show favorable outcomes.

What is claimed is:

1. A method of selecting universal donor NK cells for therapeutic administration to a subject in need thereof, the method comprising:
determining the presence of one or more variably inherited inhibitory KIRs 2DL1, 2DL2, 2DL3, and 3DL1 among the population of NK cells; and
selecting candidate NK cells as universal donor NK cells for therapeutic administration when at least one or more variably inherited inhibitory KIRs 2DL1, 2DL2, 2DL3, and 3DL1 are present in the NK cells.

2. The method of claim 1, wherein determining the presence of one or more variably inherited inhibitory KIRs 2DL1, 2DL2, 2DL3, and 3DL1 among the population of NK cells comprises:
determining a KIR phenotype of candidate NK cells from an NK cell donor, wherein the KIR phenotype is indicative of the presence of one or more variably inherited inhibitory KIRs 2DL1, 2DL2, 2DL3, and 3DL1 among the population of NK cells; or
obtaining a HLA genotype of candidate NK cells from an NK cell donor, wherein the HLA genotype is indicative of the presence of at least two HLA C1, C2, and Bw4 alleles, and thereby indicative of the presence of one or more variably inherited inhibitory KIRs 2DL1, 2DL2, 2DL3, and 3DL1 among the population of NK cells.

3. The method of claim 1, further comprising:
obtaining a KIR genotype of the candidate NK cells, wherein the KIR genotype is indicative of the presence or absence of at least three activating KIRs selected from the group consisting of 2DS1/2, 2DS3/5, 3DS1, and 2DS4, wherein selecting the candidate NK cells as universal donor NK cells further comprises selecting the candidate NK cells comprising at least three of the activating KIRs 2DS1/2, 2DS3/5, 3DS1, and/or 2DS4.

4. The method of claim 1, wherein the selected universal donor NK cells are histologically optimized for at least 50%-85% of recipient subjects.

5. The method of claim 1, further comprising:
obtaining or having obtained the CMV seropositivity of the candidate NK cells, wherein selecting a candidate NK cell as a universal donor NK cell further comprises selecting a candidate NK cell seropositive for CMV or having high NKG2C expression compared to a reference level of NKG2C expression.

6. A method of selecting universal donor NK cells for therapeutic administration to a subject in need thereof, the method comprising:
obtaining a KIR genotype of candidate NK cells, wherein the KIR genotype is indicative of the presence or absence of at least three activating KIRs selected from the group consisting of 2DS1/2, 2DS3/5, 3DS1, and 2DS4; and
selecting the candidate NK cells as universal donor NK cells for therapeutic administration when the KIR genotype indicates the presence of at least three of the activating KIRs 2DS1/2, 2DS3/5, 3DS1, and/or 2DS4; and
optionally further comprising determining the presence of one or more variably inherited inhibitory KIRs 2DL1, 2DL2, 2DL3, and 3DL1 among the population of NK cells, and further selecting candidate NK cells as universal donor NK cells for therapeutic administration when at least one or more variably inherited inhibitory KIRs 2DL1, 2DL2, 2DL3, and 3DL1 are also present in the NK cells.

7. The method of claim 6, comprising determining the presence of one or more variably inherited inhibitory KIRs 2DL1, 2DL2, 2DL3, and 3DL1 among the population of NK cells, wherein the determining comprises:
determining a KIR phenotype of candidate NK cells from an NK cell donor, wherein the KIR phenotype is indicative of the presence of one or more variably inherited inhibitory KIRs 2DL1, 2DL2, 2DL3, and 3DL1 among the population of NK cells; or
obtaining a HLA genotype of candidate NK cells from an NK cell donor, wherein the HLA genotype is indicative of the presence of at least two HLA C1, C2, and Bw4 alleles, and thereby indicative of the presence of one or more variably inherited inhibitory KIRs 2DL1, 2DL2, 2DL3, and 3DL1 among the population of NK cells.

8. A method of screening a population of candidate NK cells from a donor to identify universal NK donor cells in the population for providing a source of NK cells for therapeutic administration to subjects in need thereof, the method comprising:
determining a KIR phenotype of candidate NK cells from an NK cell donor, wherein the KIR phenotype is indicative of the presence of one or more variably inherited inhibitory KIRs 2DL1, 2DL2, 2DL3, and 3DL1 among the population of NK cells; or
obtaining a HLA genotype of candidate NK cells from an NK cell donor, wherein the HLA genotype is indicative of the presence of at least two HLA C1, C2, and Bw4 alleles, and thereby indicative of the presence of one or more variably inherited inhibitory KIRs 2DL1, 2DL2, 2DL3, and 3DL1 among the population of NK cells;
wherein candidate NK cells comprising at least two variably inherited inhibitory KIRs 2DL1, 2DL2, 2DL3, and 3DL1 are identified as universal donor NK cells.

9. The method of claim 8, further comprising:
obtaining a KIR genotype of the candidate NK cells, wherein the KIR genotype is indicative of the presence or absence of at least three activating KIRs selected from the group consisting of 2DS1/2, 2DS3/5, 3DS1, and 2DS4;
wherein candidate NK cells comprising at least three activating KIRs 2DS1/2, 2DS3/5, 3DS1, and/or 2DS4 are further identified as universal donor NK cells.

10. The method of claim 8, wherein the selected universal donor NK cells are histologically optimized for at least 50%-85% of recipient subjects.

11. The method of claim 8, further comprising obtaining or having obtained the CMV seropositivity of the candidate NK cells, wherein selecting a candidate NK cell as a universal donor NK cell further comprises selecting a candidate NK cell seropositive for CMV or having high NKG2C expression compared to a reference level of NKG2C expression.

12. An isolated universal donor NK cell selected by the method of claim 1.

13. The isolated universal donor NK cell of claim 12, wherein the NK cells are NKG2C+.

14. The isolated universal donor NK cell of claim 12, incubated in vitro in the presence of IL-21.

15. The isolated universal NK cell of claim 14, wherein the IL-21 comprises at least one of soluble IL-21, IL-21-expressing feeder cells (FC21), IL-21 plasma membrane particles (PM21s), and IL-21 exosomes (EX21s).

16. A method of treating a cancer or an infectious disease in a subject comprising administering to the subject a universal donor NK cell selected by the method of claim 1.

17. The method of claim 16, wherein the cancer is selected from a cancer of the blood, lung, esophagus, stomach, pancreas, liver, biliary tract, colon, rectum, breast, ovary, cervix uterus, endometrium, kidney, bladder, testes, prostate, larynx, thyroid, brain or skin.

18. The method of claim 16, wherein the infectious disease is caused by a pathogen selected from a virus, bacterium or fungus.

19. A method for preparing a population of universal donor NK cells for therapeutic administration to a subject in need thereof, the method comprising: (a) selecting universal donor NK cells by the method of claim 1 to obtain an initial population of universal donor NK cells; and (b) exposing the initial population of NK cells to IL-21 in vitro for a time and under conditions sufficient to expand the initial population of NK cells.

20. The method of claim 19, wherein the NK cell donor further has a CMV seropositive profile indicative of the presence of NKG2C+NK cells.

21. The method of claim 19, wherein exposing the initial population of NK cells to IL-21 comprises contacting the NK cells in vitro with at least one of soluble IL-21, IL-21-expressing feeder cells (FC21), IL-21 plasma membrane particles (PM21s) and IL-21 exosomes (EX21s).

22. A population of universal donor NK cells prepared by the method of claim 19.

23. The method of claim 19, wherein the population of universal donor NK cells are histologically optimized for at least 50%-85% of recipient subjects.

24. The method of claim 19, further comprising obtaining or having obtained the CMV seropositivity of the candidate NK cells, wherein selecting a candidate NK cell as a universal donor NK cell to obtain the initial population of universal donor NK cells further comprises selecting a candidate NK cell seropositive for CMV or having high NKG2C expression compared to a reference level of NKG2C expression.
